(19) United States
(12) Patent Application Publication
(10) Pub. No.: US 2019/0194143 A1
(43) Pub. Date: Jun. 27, 2019

(54) GLUCAGON RECEPTOR ANTAGONISTS

(71) Applicant: Ligand Pharmaceuticals, Inc., San Diego, CA (US)

(72) Inventors: Lin Zhi, San Diego, CA (US); Matthew P. Grote, San Diego, CA (US); Raja K. Reddy, San Diego, CA (US); Wenyu Li, San Diego, CA (US); William Craigo, San Diego, CA (US)

(21) Appl. No.: 16/323,508

(22) PCT Filed: Aug. 15, 2017
(86) PCT No.: PCT/US17/47031

§ 371 Date: Feb. 5, 2019

Related U.S. Application Data
(60) Provisional application No. 62/376,324, filed on Aug. 17, 2016.

(51) Int. Cl. C07D 235/16; C07D 233/61; C07D 405/10; C07D 209/18; C07D 231/12; C07D 213/81; C07D 409/06; C07D 333/38; C07D 409/12; A61P 3/10

(52) U.S. Cl. CPC C07D 235/16; C07D 233/61; C07D 405/10; C07D 209/18; A61P 3/10; C07D 213/81; C07D 409/06; C07D 333/38; C07D 409/12; C07D 231/12

(57) ABSTRACT

Provided herein are compounds, including enantiomerically pure forms thereof, and pharmaceutically acceptable salts or co-crystals and prodrugs thereof which have glucagon receptor antagonist or inverse agonist activity. Further, provided herein are pharmaceutical compositions and methods of treating, preventing, delaying the time to onset or reducing the risk for the development or progression of a disease or condition for which one or more glucagon receptor antagonist is indicated, including Type I and II diabetes, insulin resistance and hyperglycemia.

### FIELD

Provided are compounds capable of acting as antagonists of receptors. Some embodiments relate to antagonists of glucagon receptors. In some embodiments, sulfonate compounds and compositions are provided for use in treatment, prevention or amelioration of one or more symptoms, causes or effects of a glucoregulatory or glucagon receptor-mediated disease or disorder.

### BACKGROUND

Glucagon is believed to be a 29-amino acid pancreatic hormone which is secreted from the pancreatic α cells into the portal blood supply in response to hypoglycemia. It has been observed acting as a counterregulatory hormone to insulin. Some physiological effects of glucagon are mediated by its interaction with glucagon receptors, followed by activation of adenylate cyclase to increase intracellular cAMP levels. The observed result is an increase in glycogenolysis and gluconeogenesis, with attenuations of the ability of insulin to inhibit these metabolic processes. Overall rates of hepatic glucose synthesis and glycogen metabolism may be controlled by the systemic ratio of insulin and glucagon.

Diabetes is a disease characterized by elevated levels of plasma glucose. Uncontrolled hyperglycemia is associated with an increased risk for microvascular and macrovascular diseases, including nephropathy, retinopathy, hypertension, stroke, and heart disease, for example. Control of glucose homeostasis is a major approach to the treatment of diabetes. It has been demonstrated in healthy animals as well as in animal models of types I and II diabetes that removal of circulating glucagon with selective and specific antibodies results in reduction of the glycemic level. One potential treatment for diabetes and other diseases involving impaired glycemia is to block a glucagon receptor with a glucagon receptor antagonist to improve insulin responsiveness, and decrease the rate of gluconeogenesis, and/or to lower plasma glucose levels by reducing the rate of hepatic glucose output in a patient.

Some glucagon receptor antagonists that demonstrate highly potent biological activities are carboxylic compounds and bind to glucagon receptors at an allosteric site at the edge of transmembrane. The carboxylic group of the antagonists plays a role in the binding affinity and the conformation of the glucagon receptor, where the two oxygen atoms of the carboxylic acid have hydrogen bond interactions with the receptor based on the bond distances in the resolved X-ray structure of glucagon receptor and the inhibitor compound MK-0893. Sulfonate compounds have chemical and physical properties that are quite different from the carboxylate compounds and, as a result, sulfonic acid is note generally considered as a biostere of carboxylic acid by medicinal chemists. Not all compounds that are glucagon antagonists have characteristics affording the best potential to become useful therapeutics. Some of these characteristics include high affinity at the glucagon receptor, certain conformations of the antagonized glucagon receptor, duration of receptor deactivation, oral bioavailability, tissue distribution, and stability (e.g., ability to formulate or crystallize, shelf life). Favorable characteristics can lead to improved safety, tolerability, efficacy, therapeutic index, patient compliance, cost efficiency, manufacturing ease, etc. All documents referred to herein are incorporated by reference into the present application as though fully set forth herein.

### SUMMARY

Provided herein are compounds, including enantiomerically pure and substantially enantiomerically pure forms thereof, and pharmaceutically acceptable salts or co-crystals and prodrugs thereof which have glucagon receptor antagonist or inverse agonist activity. Further, provided herein are pharmaceutical compositions comprising the same, as well as methods of treating, preventing, delaying the time to onset or reducing the risk for the development or progression of a disease or condition for which one or more glucagon receptor antagonist is indicated, including without limitation Type I and II diabetes, insulin resistance and hyperglycemia. Moreover, provided herein are methods of making or manufacturing compounds disclosed herein, including enantiomerically pure forms thereof, and pharmaceutically acceptable salts or co-crystals and prodrugs thereof. It has been unexpectedly discovered that specific stereochemistry and functional groups of the compounds of the present embodiments exhibit one or more desired characteristics, including markedly improved receptor binding properties, oral bioavailability, and/or other advantageous features that enhance their suitability for therapeutic use.

In one aspect, a compound of Formula I is provided:

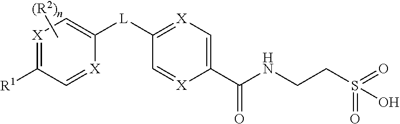

wherein:

R¹ is selected from the group consisting of hydrogen, halogen, an optionally substituted C₁₋₆-alkyl, an optionally substituted C₁₋₆-heteroalkyl, an optionally substituted C₂₋₆-alkenyl, an optionally substituted C₁₋₆-heteroalkenyl, an optionally substituted aryl, and an optionally substituted heteroaryl;

R² is selected from the group consisting of hydrogen, halogen, and an optionally substituted C₁₋₆-alkyl;

R³ is selected from the group consisting of hydrogen, an optionally substituted C₁₋₆-alkyl, and an optionally substituted C₁₋₆-heteroalkyl;

X is independently CH or N;

L is selected from a group consisting of —OCHR³—, —SCHR³—, —NR³CHR³—, —CHR¹CHR³—, —CHR³O—, —CHR³S—, —CHR³NR³—, -arylO—, -heteroarylCHR³—, —heteroarylCHR³NH—, -heteroarylCHR³O—, -heteroarylO—, —CHR³aryl-, —CHR³heteroaryl—, —OCH₂CHR³—, —NHCHR³aryl-, —OCHR³aryl-, and —NHCHR³heteroaryl-, where the aryl and the heteroaryl are independently optionally substituted;

n is 1, 2, or 3;

or a pharmaceutically acceptable salt, solvate, or prodrug thereof.

In another aspect, a compound of Formula II is provided:

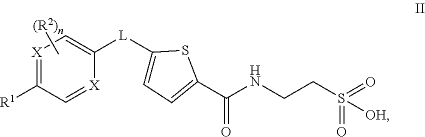

wherein:

R¹ is selected from the group consisting of hydrogen, halogen, an optionally substituted C₁₋₆-alkyl, an optionally substituted C₁₋₆-heteroalkyl, an optionally substituted C₂₋₆-alkenyl, an optionally substituted C₁₋₆-heteroalkenyl, an optionally substituted aryl, and an optionally substituted heteroaryl;

R² is selected from the group consisting of hydrogen, halogen, and an optionally substituted C₁₋₆-alkyl;

R³ is selected from the group consisting of hydrogen, an optionally substituted C₁₋₆-alkyl, and an optionally substituted C₁₋₆-heteroalkyl;

X is independently CH or N;

L is selected from a group consisting of —OCHR³—, —SCHR³—, —NR³CHR³—, —CHR¹CHR³—, —CHR³O—, —CHR³S—, —CHR³NR³—, -arylCHR³—, -arylO—, -heteroarylCHR³—, —heteroarylCHR³NH—, -heteroarylCHR³O—, -heteroarylO—, —CHR³aryl-, —CHR³heteroaryl-, —OCH₂CHR³—, —NHCHR³aryl-, —OCHR³aryl-, and —NHCHR³heteroaryl-, where the aryl and the heteroaryl are independently optionally substituted;

n is 1, 2, or 3;

or a pharmaceutically acceptable salt, solvate, or prodrug thereof.

In another aspect, pharmaceutical compositions are provided comprising a compound provided herein, e.g., a compound of Formula I or II, including a single enantiomer, a mixture of enantiomers, or a mixture of diastereomers thereof, a mixture of compounds of Formula I and II, or a pharmaceutically acceptable salt, solvate, or prodrug thereof; in combination with one or more pharmaceutically acceptable carriers.

Further provided herein is a method of treating, preventing, or ameliorating one or more symptoms of a condition, disorder, or disease associated with a glucagon receptor, comprising administering to a subject having, showing symptoms of or being suspected to have such a condition, disorder, or disease, a therapeutically effective amount of a compound provided herein, e.g., a compound of Formula I or II (or a combination of both), or a pharmaceutically acceptable salt, solvate, or prodrug thereof.

Additionally, provided herein is a method of treating, preventing, or ameliorating one or more symptoms of a condition, disorder, or disease responsive to the modulation of a glucagon receptor (GCGR), comprising administering to a subject having, showing symptoms of, or being suspected to have such a condition, disorder, or disorder, a therapeutically effective amount of a compound provided herein, e.g., a compound of Formula I or II (or a combination thereof), or a pharmaceutically acceptable salt, solvate, or prodrug thereof; or a pharmaceutical composition thereof.

Also provided herein is a method of treating, preventing, or ameliorating one or more symptoms of a GCGR-mediated condition, disorder, or disease, comprising administering to a subject having or being suspected to have such a condition, disorder, or disease, a therapeutically effective amount of a compound provided herein, e.g., a compound of Formula I or II (or a combination thereof), or a pharmaceutically acceptable salt, solvate, or prodrug thereof; or a pharmaceutical composition thereof.

Further provided herein is a method of treating, preventing, or ameliorating one or more symptoms of a condition, disorder, or disease responsive to a decrease in the hepatic glucose production or in the blood glucose level of a subject, comprising administering to the subject a therapeutically effective amount of a compound provided herein, e.g., a compound of Formula I or II (or a combination thereof), or a pharmaceutically acceptable salt, solvate, or prodrug thereof; or a pharmaceutical composition thereof.

Additionally provided herein is a method of modulating the biological activity of a glucagon receptor, comprising contacting the receptor with one or more of the compounds provided herein, e.g., a compound of Formula I or II (or a combination thereof), or a pharmaceutically acceptable salt, solvate, or prodrug thereof; or a pharmaceutical composition thereof.

These and other aspects of the present embodiments will be more clearly understood with reference to the following detailed description.

### DETAILED DESCRIPTION

Definitions

To facilitate understanding of the disclosure set forth herein, a number of terms are defined below.

Generally, the nomenclature used herein and the laboratory procedures in organic chemistry, medicinal chemistry, and pharmacology described herein are those well-known and commonly employed in the art. Unless defined otherwise, all technical and scientific terms used herein generally have the same meaning as commonly understood by one of ordinary skill in the art to which this disclosure belongs.

The term “subject” refers to an animal, including, but not limited to, a primate (e.g., human), cow, sheep, goat, horse, dog, cat, rabbit, rat, or mouse. The terms “subject” and “patient” are used interchangeably herein in reference, for example, to a mammalian subject, such as a human subject.

The terms “treat,” “treating,” and “treatment” are meant to include alleviating or abrogating a disorder, disease, or condition, or one or more of the symptoms associated with the disorder, disease, or condition; or alleviating or eradicating the cause(s) of the disorder, disease, or condition itself.

The terms “prevent,” “preventing,” and “prevention” are meant to include a method of delaying and/or precluding the onset of a disorder, disease, or condition, and/or its attendant symptom(s); barring a subject from acquiring a disease; or reducing a subject's risk of acquiring a disorder, disease, or condition.

The term “therapeutically effective amount” is meant to include the amount of a compound that, when administered, is sufficient to prevent development of, or alleviate to some extent, one or more of the symptoms of a disorder, disease, or condition being treated. The term “therapeutically effective amount” also refers to the amount of a compound that is sufficient to elicit the biological or medical response of a cell, tissue, system, animal, or human, which is being sought by a researcher, veterinarian, medical doctor, or clinician.

The term “IC₅₀” refers an amount, concentration, or dosage of a compound that is required for 50% inhibition of a maximal response in an assay that measures such response.

The term “pharmaceutically acceptable carrier,” “pharmaceutically acceptable excipient,” “physiologically acceptable carrier,” or “physiologically acceptable excipient” refer to a pharmaceutically-acceptable material, composition, or vehicle, such as a liquid or solid filler, diluent, excipient, solvent, or encapsulating material. In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, Remington: The Science and Practice of Pharmacy, 21st Edition, Lippincott Williams & Wilkins: Philadelphia, Pa., 2005; Handbook of Pharmaceutical Excipients, 5th Edition, Rowe et al., Eds., The Pharmaceutical Press and the American Pharmaceutical Association: 2005; and Handbook of Pharmaceutical Additives, 3rd Edition, Ash and Ash Eds., Gower Publishing Company: 2007; Pharmaceutical Preformulation and Formulation, Gibson Ed., CRC Press LLC: Boca Raton, Fla., 2004 (incorporated herein by reference).

The term “about” or “approximately” means an acceptable error for a particular value as determined by one of ordinary skill in the art, which depends in part on how the value is measured or determined. In certain embodiments, the terra “about” or “approximately” means within 1, 2, 3, or 4 standard deviations. In certain embodiments, the term “about” or “approximately” means within 50%, 20%, 15%, 10%, 9%, 8%, 7%, 6%, 5%, 4%, 3%, 2%, 1%, 0.5%, or 0.05% of a given value or range.

The terms “active ingredient” and “active substance” refer to a compound, which is administered, alone or in combination with one or more pharmaceutically acceptable excipients, to a subject for treating, preventing, or ameliorating one or more symptoms of a condition, disorder, or disease. As used herein, “active ingredient” and “active substance” may be an optically active isomer of a compound described herein.

The terms “drug,” “therapeutic agent,” and “chemotherapeutic agent” refer to a compound, or a pharmaceutical composition thereof, which is administered to a subject for treating, preventing, or ameliorating one or more symptoms of a condition, disorder, or disease.

The term “naturally occurring” or “native” when used in connection with biological materials such as nucleic acid molecules, polypeptides, host cells, and the like, refers to materials which are found in nature and are not manipulated by humans. Similarly, “non-naturally occurring” or “non-native” refers to a material that is not found in nature or that has been structurally modified or synthesized by humans.

The term “glucagon receptor” or “GCGR” refers to a glucagon receptor protein or variant thereof, which is capable of mediating a cellular response to glucagon in vitro or in vivo. GCGR variants include proteins substantially homologous to a native GCGR, e.g., proteins having one or more naturally or non-naturally occurring amino acid deletions, insertions, or substitutions (e.g., GCGR derivatives, homologs, and fragments), as compared to the amino acid sequence of a native GCGR. In certain embodiments, the amino acid sequence of a GCGR variant is at least about 80% identical, at least about 90% identical, or at least about 95% identical to a native GCGR. In certain embodiments, the GCGR is a human glucagon receptor.

The term “glucagon receptor antagonist” or “GCGR antagonist” refers to a compound that, e.g., partially or completely blocks, decreases, prevents, inhibits, or downregulates GCGR activity. These terms also refer to a compound that binds to, delays the activation of, inactivates, or desensitizes GCGR. A GCGR antagonist may act by interfering with the interaction of glucagon with GCGR.

The term “GCGR-mediated condition, disorder, or disease” refers to a condition, disorder, or disease characterized by inappropriate, e.g., less than or greater than normal, GCGR activity. Inappropriate GCGR functional activity might arise as the result of an increase in glucagon concentration, GCGR expression in cells which normally do not express GCGR, increased GCGR expression or degree of intracellular activation, leading to, e.g., abnormal plasma glucose levels; or decreased GCGR expression. A GCGR-mediated condition, disorder or disease may be completely or partially mediated by inappropriate GCGR activity. A GCGR-mediated condition, disorder or disease is one in which modulation of GCGR results in some effect on the underlying symptom, condition, disorder, or disease, e.g., a GCGR antagonist results in some improvement in at least some of patients being treated.

The term “alkyl” and the prefix “alk” refers to a linear or branched saturated monovalent hydrocarbon radical, wherein the alkyl may optionally be substituted with one or more substituents. The term “alkyl” also encompasses linear, branched, and cyclic alkyl groups, unless otherwise specified. In certain embodiments, the alkyl is a linear saturated monovalent hydrocarbon radical that has 1 to 20 (C₁₋₂₀), 1 to 15 (C₁₋₁₅), 1 to 12 (C₁₋₁₂), 1 to 10 (C₁-10), or 1 to 6 (C₁₋₆) carbon atoms, or branched saturated monovalent hydrocarbon radical of 3 to 20 (C₃₋₂₀), 3 to 15 (C₃₋₁₅), 3 to 12 (C₃₋₁₂), 3 to 10 (C₃₋₁₀), or 3 to 6 (C₃₋₆) carbon atoms. As used herein, linear C₁₋₆ and branched C₃₋₆ alkyl groups are also referred as “lower alkyl,” Examples of alkyl groups include, but are not limited to, methyl, ethyl, propyl (including all isomeric forms), n-propyl, isopropyl, butyl (including all isomeric forms), n-butyl, isobutyl, t-butyl, pentyl (including all isomeric forms), and hexyl (including all isomeric forms). For example, C₁₋₆ alkyl refers to a linear saturated monovalent hydrocarbon radical of 1 to 6 carbon atoms or a branched saturated monovalent hydrocarbon radical of 3 to 6 carbon atoms. Cycloalkyl also includes monocyclic rings fused to an aryl group in which the point of attachment is on the non-aromatic portion. Examples of cycloalkyl include cyclopropyl, cyclobutyl, cyclopentyl, cyclohexyl, cycloheptyl, tetrahydronaphthyl, decahydronaphthyl, indanyl and the like.

The term “alkenyl” refers to a linear or branched monovalent hydrocarbon radical, which contains one or more, in one embodiment, one to five, carbon-carbon double bonds. The alkenyl may be optionally substituted with one or more substituents. The term “alkenyl” also embraces radicals having “cis” and “trans” configurations, or alternatively, “E” and “Z” configurations, as appreciated by those of ordinary skill in the art. As used herein, the term “alkenyl” encompasses both linear and branched alkenyl, unless otherwise specified. For example, C₂₋₆ alkenyl refers to a linear unsaturated monovalent hydrocarbon radical of 2 to 6 carbon atoms or a branched unsaturated monovalent hydrocarbon radical of 3 to 6 carbon atoms. In certain embodiments, the alkenyl is a linear monovalent hydrocarbon radical of 2 to 20 (C₂₋₂₀), 2 to 15 (C₂₋₁₅), 2 to 12 (C₂₋₁₂), 2 to 10 (C₇₋₁₀), or 2 to 6 (C₂₋₆) carbon atoms, or a branched monovalent hydrocarbon radical of 3 to 20 (C₃₋₂₀), 3 to 15 (C₃₋₁₅), 3 to 12 (C₃₋₁₂), 3 to 10 (C₃₋₁₀), or 3 to 6 (C₃₋₆) carbon atoms. Examples of alkenyl groups include, but are not limited to, vinyl, isopropenyl, pentenyl, hexenyl, heptenyl, ethenyl, propen-1-yl, propen-2-yl, allyl, butenyl, 2-butenyl, 2-methyl-2-butenyl, 4-methylbutenyl, and the like.

The term “cycloalkyl” refers to a cyclic saturated bridged and/or non-bridged monovalent hydrocarbon radical, which may be optionally substituted with one or more substituents. In certain embodiments, the cycloalkyl has from 3 to 20 (C₃₋₂₀), from 3 to 15 (C₃₋₁₅), from 3 to 12 (C₃₋₁₂), from 3 to 10 (C₃₋₁₀), or from 3 to 7 (C₃₋₇) carbon atoms. Examples of cycloalkyl groups include, but are not limited to, cyclopropyl, cyclobutyl, cyclopentyl, cyclohexyl, cycloheptyl, decalinyl, and adamantyl. Cycloalkyl also includes monocyclic rings fused to an aryl group in which the point of attachment is on the non-aromatic portion. Additional examples of cycloalkyl include tetrahydronaphthyl, decahydronaphthyl, indanyl and the like.

The term “aryl” (Ar) refers to a monocyclic aromatic group and/or multicyclic monovalent aromatic group that contain at least one aromatic hydrocarbon ring. In certain embodiments, the aryl has from 6 to 20 (C₆₋₂₀), from 6 to 15 (C₆₋₁₅), or from 6 to 10 (C₆₋₁₀) ring atoms. In some embodiments, the aryl has from 5 to 20 (C₆₋₂₀), from 5 to 15 (C₆₋₁₅), or from 5 to 10 (C₆₋₁₀) ring atoms. In some embodiments, the aryl has from 4 to 20 (C₆₋₂₀), from 4 to 15 (C₆₋₁₅), or from 4 to 10 (C₆₋₁₀) ring atoms. Examples of aryl groups include, but are not limited to, phenyl, naphthyl, fluorenyl, azulenyl, anthryl, phenanthryl, pyrenyl, biphenyl, and terphenyl. Aryl also refers to bicyclic or tricyclic carbon rings, where one of the rings is aromatic and the others of which may be saturated, partially unsaturated, or aromatic, for example, dihydronaphthyl, indenyl, indanyl, or tetrahydronaphthyl (tetralinyl). In certain embodiments, aryl may also be optionally substituted with one or more substituents.

The term “heteroaryl” refers to a monocyclic aromatic group and/or multicyclic aromatic group that contain at least one aromatic ring, wherein at least one aromatic ring contains one or more heteroatoms independently selected from O, S, and N. In some embodiments, each ring contains 5 to 6 atoms. In some embodiments, each ring of a heteroaryl group can contain one or two O atoms, one or two S atoms, and/or one to four N atoms, provided that the total number of heteroatoms in each ring is four or less and each ring contains at least one carbon atom. The heteroaryl may be attached to the main structure at any heteroatom or carbon atom which results in the creation of a stable compound. In certain embodiments, the heteroaryl has from 5 to 20, from 5 to 15, or from 5 to 10 ring atoms. Examples of monocyclic heteroaryl groups include, but are not limited to, pyrrolyl, pyrazolyl, pyrazolinyl, imidazolyl, oxazolyl, isoxazolyl, thiazolyl, thiadiazolyl, isothiazolyl, furanyl, thienyl, oxadiazolyl, pyridyl, pyrazinyl, pyrirnidinyl, pyramidyl, pyridazinyl, triazolyl, tetrazolyl, and triazinyl. Examples of bicyclic heteroaryl groups include, but are not limited to, indolyl, benzothiazolyl, benzoxazolyl, benzothienyl, benzothiophenyl, furo(2,3-b) pyridyl, quinolinyl, tetrahydroisoquinolinyl, isoquinolyl, isoquinolinyl, benzimidazolyl, benzopyranyl, indolizinyl, benzofuranyl, isobenzofuranyl, chromonyl, coumarinyl, cinnolinyl, quinoxalinyl, indazolyl, purinyl, pyrrolopyridinyl, tbropyridinyl, thienopyridinyl, dihydroisoindolyl, and tetrahydroquinolinyl. Examples of tricyclic heteroaryl groups include, but are not limited to, carbazotyl, benzindolyl, phenanthrollinyl, acridinyl, phenanthridinyl, and xanthenyl. In certain embodiments, heteroaryl may also be optionally substituted with one or more substituents. Heteroaryl also includes aromatic heterocyclic groups fused to heterocycles that are non-aromatic or partially aromatic, and aromatic heterocyclic groups fused to cycloalkyl rings. Heteroaryl also includes such groups in charged form, e.g., pyridinium.

The term “heteroaryl-alkyl” refers to a monovalent alkyl group substituted with heteroaryl. In certain embodiments, both alkyl and heteroaryl may be optionally substituted with one or more substituents.

The term “heterocyclyl” (Hetcy) or “heterocycle” refers to a monocyclic non-aromatic ring system and/or multicyclic ring system that contains at least one non-aromatic ring, wherein one or more of the non-aromatic ring atoms are heteroatoms independently selected from O, S, or N; and the remaining ring atoms are carbon atoms. In certain embodiments, the heterocyclyl or heterocyclic group has from 3 to 20, from 3 to 15, from 3 to 10, from 3 to 8, from 4 to 7, or from 5 to 6 ring atoms. In certain embodiments, the heterocyclyl is a monocyclic, bicyclic, tricyclic, or tetracyclic ring system, which may include a fused or bridged ring system, and in which the nitrogen or sulfur atoms may be optionally oxidized, the nitrogen atoms may be optionally quaternized and some rings may be partially or fully saturated, or aromatic. The heterocyclyl may be attached to the main structure at any heteroatom or carbon atom which results in the creation of a stable compound. Examples of such heterocyclic radicals include, but are not limited to benzoxazinyl, benzodioxanyl, benzodioxolyl, benzopyranonyl, benzopyranyl, benzotetrahydrofuranyl, benzotetrahydrothienyl, benzothiopyranyl, chromanyl, chromonyl, coumarinyl, decahydroisoquinolinyl, dihydrobenzisothiazinyl, dihydrobenzisoxazinyl, 2,3-dihydrofuro(2,3-b)pyridyl, dihydrofuryl, dihydroindotyl, dihydropyranyl, dioxolanyl, dihydropyrazinyl, dihydropyridinyl, dihydropyrazolyl, dihydropyrimidinyl, dihydropyrrolyl, 1,4-dithianyl, imidazolidinyl, imidazolinyl indolinyl, isobenzotetrahydrofuranyl, isobenzotetrahydrothienyl, isochromanyl, isocoumarinyl, isoindolinyl, isothiazolidinyl, isoxazolidinyl, morpholinyl, octahydroindolyl, octahydroisoindolyl, oxazolidinonyl, oxazolidinyl, oxiranyl, quinuclidinyl, tetrahydrofuryl, tetrahydrofuranyl, tetrahydrohydroquinolinyl, tetrahydroisoquinolinyl, tetrahydropyranyl, tetrahydrothienyl, thiamorpholinyl, thiazolidinyl, and 1,3,5-trithianyl. Heterocyclyl/heterocyclic also includes partially unsaturated monocyclic rings that are not aromatic, such as 2- or 4-pyridones attached through the nitrogen or N-substituted-(1H,3H)-pyrimidine -2,4-diones (N-substituted uracils). Heterocyclyl/heterocyclic also includes such moieties in charged form, e.g., piperidinium. In certain embodiments, heterocyclyl/heterocyclic may also be optionally substituted with one or more substituents.

The term “alkoxy” refers to an —OR radical, wherein R is, for example, alkyl, alkenyl, alkynyl, cycloalkyl, aryl, heteroaryl, or heterocyclyl, each as defined herein. When R is aryl, it is also known as aryloxy. Examples of alkoxy groups include, but are not limited to, methoxy, ethoxy, propoxy, n-propoxy, 2-propoxy, n-butoxy, isobutoxy, tert-butoxy, cyclohexyloxy, phenoxy, benzoxy, and 2-naphthyloxy. In certain embodiments, alkoxy may also be optionally substituted with one or more substituents.

The term “acyl” refers to a —C(O)R radical, wherein R is, for example, hydrogen, alkyl, alkenyl, alkynyl, cycloalkyl, aryl, heteroaryl, or heterocyclyl, each as defined herein. Examples of acyl groups include, but are not limited to, formyl, acetyl, propionyl, butanoyl, isobutanoyl, pentanoyl, hexanoyl, heptanoyl, octanoyl, nonanoyl, decanoyl, dodecanoyl, tetradecanoyl, hexadecanoyl, octadecanoyl, eicosanoyl, docosanoyl, myristoleoyl, palmitoleoyl, oleoyl, linoleoyl, arachidonoyl, benzoyl, pyridinylcarbonyl, and furoyl. In certain embodiments, acyl may also be optionally substituted with one or more substituents.

The term “halogen”, “halide” or “halo” (Halo) refers to fluorine, chlorine, bromine, and/or iodine.

The term “optionally substituted” is intended to mean that a group, including alkyl, alkoxy, acyl, alkyl-cycloalkyl, hydroxyalkyl, alkenyl, alkynyl, cycloalkyl, cycloalkenyl, cycloalkynyl, cycloalkylalkyl, aryl, aryloxy, aralkyl, aryl-alkenyl, aryl-alkynyl, heteroaryl, heteroarylalkyl, heteroaryl-alkenyl, heteroaryl-alkynyl, and heterocyclyl, or acyl, may be substituted with one or more substituents, in one embodiment, one, two, three, four substituents, Where in some embodiments each substituent is independently selected from the group consisting of cyano, halo, oxo, nitro, C₁₋₆ alkyl, C₂₋₆ alkenyl, C₂₋₆ alkynyl, C₃₋₇ cycloalkyl, C₆₋₁₄ aryl, heteroaryl (wherein each aryl and heteroaryl are independently optionally substituted with halogen, alkyl, heteroalkyl, haloalkyl, and alkyloxy), heterocyclyl, —C(O)R^{e}, —C(O)OR^{e}, —C(O)NR^{f}R^{g}, —C(NR^{e})NR^{f}R^{g}, —OR^{e}, —OC(O)R^{e}, —OC(O)OR^{e}, —OC(O)NR^{f}R^{g}, —OC(═NR^{e})NR^{f}R^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{f}, —NR^{e}C(O)OR^{f}, —NR^{e}C(O)NR^{f}R^{g}, —NR^{e}C(═NR^{h})NR^{f}R^{g}, —NR^{e}S(O)R^{f}, —NR^{e}S(O)₂R^{f}, —NR^{e}S(O)NR^{f}R^{g}, —NR^{e}S(O)₂NR^{f}R^{g}, —SR^{e}, —S(O)R^{e}, —S(O)₂R^{e}, and —S(O)₂NR^{f}R^{g}, wherein each R^{e}, R^{f}, R^{g}, and R^{h} is independently hydrogen, C₁-₆ alkyl, C₂₋₆ alkenyl, C₂₋₆ alkynyl, C₃₋₇ cycloalkyl, C₆₋₁₄ aryl, heteroaryl, or heterocyclyl; or R^{f} and R^{g} together with the N atom to which they are attached form heterocyclyl.

The term “optically active” refers to a collection of molecules, which has an enantiomeric excess of no less than about 50%, no less than about 70%, no less than about 80%, no less than about 90%, no less than about 91%, no less than about 92%, no less than about 93%, no less than about 94%, no less than about 95%, no less than about 96%, no less than about 97%, no less than about 98%, no less than about 99%, no less than about 99.5%, or no less than about 99.8%.

In describing an optically active compound, the prefixes R and S are used to denote the absolute configuration of the molecule about its chiral center(s). The (+) and (−) are used to denote the optical rotation of the compound, that is, the direction in which a plane of polarized light is rotated by the optically active compound. The (−) prefix indicates that the compound is levorotatory, that is, the compound rotates the plane of polarized light to the left or counterclockwise. The (+) prefix indicates that the compound is dextrorotatory, that is, the compound rotates the plane of polarized light to the right or clockwise. However, the sign of optical rotation, (+) and (−), is not related to the absolute configuration of the molecule, R and S.

The term “solvate” refers to a compound provided herein or a salt thereof, which further includes a stoichiometric or non-stoichiometric amount of solvent bound by non-covalent intermolecular forces. Where the solvent is water, the solvate is a hydrate.

“Binding” means the specific association of the compound of interest to the target of interest, e.g., a receptor.

The term “crystalline” and related terms used herein, when used to describe a substance, component or product, means that the substance, component or product is crystalline as determined by X-ray diffraction. See, e.g., Remington's Pharmaceutical Sciences, 18^{th} ed, Mack Publishing, Easton, Pa., 173 (1990); The United States Pharmacopeia, 23^{rd} ed, 1843-1844 (1995) (incorporated herein by reference).

“Co-crystal” as used herein means a crystalline material comprised of two or more unique solids at room temperature that are H-bonded.

“Diabetes” refers to a heterogeneous group of disorders associated with impaired glucose tolerance. Its diagnosis and characterization, including pre-diabetes, type and type II diabetes, and a variety of syndromes characterized by impaired glucose tolerance, impaired fasting glucose, and abnormal glycosylated hemoglobin, are well known in the art. It may be characterized by hyperglycemia, glycosuria, ketoacidosis, neuropathy or nephropathy, increased hepatic glucose production, insulin resistance in various tissues, insufficient insulin secretion and enhanced or poorly controlled glucagon secretion from the pancreas.

The term “drug” refers to a compound, or a pharmaceutical composition thereof, which is administered to a subject for treating, preventing, or ameliorating one or more symptoms of a condition, disorder, or disease.

The term “EC₅₀” refers an amount, concentration, or dosage of a compound at which 50% of a maximal response is observed in an assay that measures such response.

The term “percent enantiomeric excess (% ee)” refers to optical purity. It is obtained by using the following formula:

$$\frac{[ R ] - [ S ]}{[ R ] + [ S ]} \times 100 = \% R - \% S$$

where [R] is the amount of the R isomer and [S] is the amount of the S isomer. This formula provides the % ee when R is the dominant isomer.

The term “enantiomerically pure” refers to a compound which comprises at least about 80% by weight of the designated enantiomer and at most about 20% by weight of the other enantiomer or other stereoisomer(s), at least about 90% by weight of the designated enantiomer and at most about 10% by weight of the other enantiomer or other stereoisomer(s), at least about 95% by weight of the designated enantiomer and at most about 5% by weight of the other enantiomer or other stereoisomer(s), at least about 96.6% by weight of the designated enantiomer and at most about 3.4% by weight of the other enantiomer or other stereoisomer(s), at least about 9.7% by weight of the designated enantiomer and at most about 3% by weight of the other enantiomer or other stereoisomer(s), at least about 99% by weight of the designated enantiomer and at most about 1% by weight of the other enantiomer or other stereoisomer(s), or at least about 99.9% by weight of the designated enantiomer and at most about 0.1% by weight of the other enantiomer or other stereoisomer(s). In certain embodiments, the weights are based upon total weight of the compound.

The term “chiral” as used herein includes a compound that has the property that it is not superimposable on its mirror image.

“Insulin resistance” is defined clinically as the impaired ability of a known quantity of exogenous or endogenous insulin to increase whole body glucose uptake and utilization.

“Impaired glucose tolerance (IGT)” refers to a condition known to precede the development of overt Type 2 diabetes. It is characterized by abnormal blood glucose excursions following a meal. The criteria for diagnosing and characterizing impaired glucose tolerance and related syndromes are well known in the art.

“Lower” referred to herein in connection with organic radicals or compounds respectively defines such radicals or compounds as containing up to and including 6 carbon atoms. One aspect provides organic radicals or compounds as containing up to and including 4 carbon atoms. Yet another aspect provides organic radicals or compounds that contain one to three carbon atoms. Such groups may be straight chain, branched, or cyclic.

“Metabolic disease” includes diseases and conditions such as obesity, diabetes and lipid disorders such as hypercholesterolemia, hyperlipidemia, hypertriglyceridemia as well as disorders that are associated with abnormal levels of lipoproteins, lipids, carbohydrates and insulin such as metabolic syndrome X, diabetes, impaired glucose tolerance, atherosclerosis, coronary artery disease, cardiovascular disease. The criteria for diagnosing and characterizing these conditions and related syndromes are well known in the art.

“Prodrug” as used herein refers to any compound that when administered to a biological system generates a biologically active compound as a result of spontaneous chemical reaction(s), enzyme catalyzed chemical reaction(s), and/or metabolic chemical reaction(s), or any combination thereof. Standard prodrugs are formed using groups attached to functionality, e.g., HO—, HS—, HOOC—, —NHR, associated with the drug, that cleave in vivo. Standard prodrugs include but are not limited to carboxylate esters where the group is alkyl, aryl, aralkyl, acyloxyalkyl, alkoxycarbonyloxyalkyl as well as esters of hydroxyl, thiol and amines where the group attached is an acyl group, an alkoxycarbonyl, aminocarbonyl, phosphate or sulfate. The groups illustrated are exemplary, not exhaustive, and one skilled in the art could prepare other varieties of prodrugs. Such prodrugs of the compounds of Formula I or II disclosed herein fall within this scope. Prodrugs must undergo some form of a chemical transformation to produce the compound that is biologically active or is a precursor of the biologically active compound. In some cases, the prodrug is biologically active, usually less than the drug itself, and serves to improve drug efficacy or safety through improved oral and/or pharmacodynamic half-life, etc. Prodrug forms of compounds may be utilized, for example, to improve bioavailability, improve subject acceptability such as by masking or reducing unpleasant characteristics such as bitter taste or gastrointestinal irritability, alter solubility such as for intravenous use, provide for prolonged or sustained release or delivery, improve ease of formulation, or provide site-specific delivery of the compound. Prodrugs are described in The Organic Chemistry of Drug Design and Drug Action, by Richard B. Silverman, Academic Press, San Diego, 1992. Chapter 8: “Prodrugs and Drug delivery Systems” pp. 352-401; Design of Prodrugs, edited by H. Bundgaard, Elsevier Science, Amsterdam, 1985; Design of Biopharmaceutical Properties through Prodrugs and Analogs, Ed. by E. B. Roche, American Pharmaceutical Association, Washington, 1977; and Drug Delivery Systems, ed. by R. L. Juliano, Oxford Univ. Press, Oxford, 1980, all of which are incorporated herein by reference.

a. Compounds

One aspect provides for compounds of Formula I,

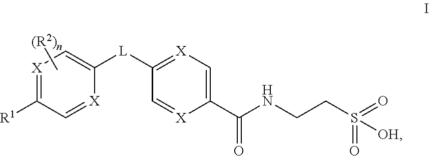

wherein:

R¹ is selected from the group consisting of hydrogen, halogen, an optionally substituted C₁₋₆-alkyl, an optionally substituted C₁₋₆-heteroalkyl, an optionally substituted C₂₋₆-alkenyl, an optionally substituted C₁₋₆-heteroalkenyl, an optionally substituted aryl, and an optionally substituted heteroaryl;

R² is selected from the group consisting of hydrogen, halogen, and an optionally substituted C₁₋₆-alkyl ;

R³ is selected from the group consisting of hydrogen, an optionally substituted C₁₋₆-alkyl, and an optionally substituted C₁₋₆-heteroalkyl;

X is independently CH or N;

L is selected from a group consisting of —OCHR³—, —SCHR³—, —CHR¹CHR³ , —CHR³O—, —CHR³S—, —CHR³NR³—, -arylCHR³—, -arylO—, -heteroarylCHR³—, -heteroarylCHR³NH—, -heteroarylCHR³O—, -heteroarylO—, —CHR³aryl-, —CHR³heteroaryl-, —OCH₂CHR³—, —NHCHR³aryl-, —OCHR³aryl-, and —NHCHR³heteroaryl-, where the aryl and the heteroaryl are independently optionally substituted;

n is 1, 2, or 3;

or a pharmaceutically acceptable salt, solvate, or prodrug thereof.

Another aspect provides compounds of Formula II:

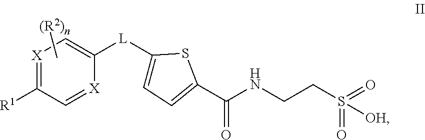

wherein:

R¹ is selected from the group consisting of hydrogen, halogen, an optionally substituted C₁₋₆-alkyl, an optionally substituted C₁₋₆-heteroalkyl, an optionally substituted C₂₋₆-alkenyl, an optionally substituted C₁₋₆-heteroalkenyl, an optionally substituted aryl, and an optionally substituted heteroaryl;

R² is selected from the group consisting of hydrogen, halogen, and an optionally substituted C₁₋₆-alkyl;

R¹ is selected from the group consisting of hydrogen, an optionally substituted C₁₋₆-alkyl, and an optionally substituted C₁₋₆-heteroalkyl;

X is independently CR or N;

L is selected from a group consisting of —OCHR³—, —SCHR³—, —NR³CHR³—, —CHR¹CHR³—, —CHR³O—, —CHR³S—, —CHR³NR³—, -arylCHR³—, -arylO—, -heteroarylCHR³—, -heteroarylCHR³NH—, —heteroarylCHR³O—, -heteroarylO—, —CHR³aryl-, —CHR³heteroaryl-, —OCH₂CHR³—, —NHCHR³aryl-, —OCHR³aryl-, and —NHCHR³heteroaryl-, where the aryl and the heteroaryl are independently optionally substituted;

n is 1, 2, or 3;

or a pharmaceutically acceptable salt, solvate, or prodrug thereof.

In other embodiments according to Formula I, R¹ is selected from a group of hydrogen, F, Cl, methyl, ethyl, tert-butyl, methoxy, trifluromethyl, an optionally substituted phenyl, an optionally substituted pyridinyl, or an optionally substituted pyrazolyl.

In another embodiment, R² is H, Cl or CH₃.

In certain embodiments, X is CH.

In certain embodiments, L is , —OCHR³—, —SCHR³—, —NR³CHR³—, —CHR¹CHR³—, —CHR³O—, —CHR³S—, —CHR³NR³—, -hetroarylCHR³—, -heteroarylO—, —OCH₂CHR³—, —NHCHR³aryl-, and —OCHR³aryl-, where the aryl and the heteroaryl are independently optionally substituted.

In other embodiments according to Formula II, R¹ is selected from a group of hydrogen, F, Cl, methyl, ethyl, tert-butyl, methoxy, trifluromethyl, an optionally substituted phenyl, an optionally substituted pyridinyl, or an optionally substituted pyrazolyl.

In another embodiment, R² is H, Cl or CH₃.

In certain embodiments, X is CH.

In certain embodiments, L is —OCHR³—, —SCHR³—, —NR³CHR³—, —CHR¹CHR³—, —CHR³O—, —CHR³S—, —CHR³NR³—, -heteroarylCHR³—, -heteroarylO—, —OCH₂CHR³—, —NHCHR³aryl-, and —OCHR³aryl-, where the aryl and the heteroaryl are independently optionally substituted.

In certain embodiments, the compound of Formula I are a racemic mixture. In certain embodiments, the compound of Formula II are a racemic mixture.

In certain embodiments, a single enantiomer is >70%, >80%, >85%, >90%, >91%, >92%, >93%, >94%, >95%, >96%, >97%, >98% or >99% as compared to the total percentage of all other enantiomers of the same compound or other diastereomers present in the composition.

Another aspect provides for salts, including pharmaceutically acceptable salts, of compounds of Formula I or II and pharmaceutical compositions comprising a pharmaceutically acceptable salt of compounds of Formula I or II. Salts of compounds of Formula I or II include an inorganic base addition salt such as for example, sodium, potassium, lithium, calcium, magnesium, ammonium, aluminum salts or organic base addition salts.

Another aspect provides for anhydrates, hydrates and solvates of compounds of Formula I or II and pharmaceutical compositions comprising a pharmaceutically acceptable anhydrates, hydrates and solvates of compounds of Formula I or II. Included are an anhydrate, hydrate or solvate of a free form or salt of a compound of Formula I or II. Hydrates include, for example, a hemihydrate, monohydrate, dihydrate, trihydrate, quadrahydrate, pentahydrate, and sesquihydrate.

Also, the activities of the compounds of Formula I or II can be described in terms of the concentrations of compounds required for displacement of 50% of the radiolabeled glucagon from the human glucagon receptor (the IC₅₀ values) according to the methods of Example A. In one embodiment, the IC₅₀ values for the compounds of Formula I are less than <10,000 nM, 9,000 nM, 8,000 nM, 7,000 nM, 6,000 nM, 5,000 nM, 4,000 nM, 3,000 nM, 2,000 nM, 1,000 nM, 900 nM, 800 nM, 700 nM, 600 nM, 500 nM, 400 nM, 300 nM, 200 nM, 100 nM, 90 nM, 80 nM, 70 nM, 60 nM, 50 nM, 40 nM, 30 nM, 25 nM, 20 nM, 15 nM, 10 nM or 5 nM.

In another alternative, the activities of the compounds of Formula I or II can be described in terms of the concentrations of compounds required for functional antagonism of glucagon in hepatocytes from various species. In one embodiment, the EC₅₀ values for the compounds of Formula I or II are less than <10,000 nM, 9,000 nM, 8,000 nM, 7,000 nM, 6,000 nM, 5,000 nM, 4,000 nM, 3,000 nM, 2,000 nM, 1,000 nM, 900 nM, 800 nM, 700 nM, 600 nM, 500 nM, 400 nM, 300 nM, 200 nM, 100 nM, 90 nM, 80 nM, 70 nM, 60 nM, 50 nM, 40 nM, 30 nM, 2.5 nM, 20 nM, 15 nM, 10 nM or 5 nM.

The compounds of Formula I or II disclosed herein also exhibit the ability to reduce blood glucose in an animal. In certain aspects, circulating blood glucose in fasting or non-fasting (freely-feeding) animals can be reduced between 10% and 100%. A reduction of 100% refers to complete normalization of blood glucose levels, not 0% blood glucose levels. Normal blood glucose in rats, for example, is approximately 80 mg/dl (fasted) and approximately 120 mg/dl (fed). Thus, contemplated herein is a method for reducing excessive circulating blood glucose levels in fasting or freely fed animals (e.g. rat), by administering, for example, 10 mg/kg of a compound of Formula I or II, by at least 10%, 20%, 30%, 40%, 50%, 60%, 70%, 80%, 90%, 95%, 99% or 100%.

b. Administration

Provided herein are pharmaceutical compositions including a compound provided herein as an active ingredient, e.g., a compound of Formula I or II, or a pharmaceutically acceptable salt, solvate, or prodrug thereof; in combination with a pharmaceutically acceptable vehicle, carrier, diluent, excipient, or a mixture thereof.

The pharmaceutical compositions may be formulated in various dosage forms, including, but not limited to, the dosage forms for oral parenteral, subcutaneous, intramuscular, transmucosal, inhaled, or topical/transdermal administration. The pharmaceutical compositions may also be formulated as modified release dosage forms, including, but not limited to, delayed-, extended-, prolonged-, sustained-, pulsatile-, controlled-, accelerated-, fast-, targeted-, programmed-release, and gastric retention dosage forms. These dosage forms can be prepared according to conventional methods and techniques known to those skilled in the art (see, Remington: The Science and Practice of Pharmacy, supra; Modified-Release Drug Deliver Technology, Rathbone et at, Eds., Drugs and the Pharmaceutical Science, Marcel Dekker, Inc.: New York, N.Y., 2003; Vol. 126) (incorporated herein by reference).

The pharmaceutical compositions provided herein may be provided in a unit- or multiple-dosage form. A unit-dosage form, as used herein, refers to a physically discrete unit suitable for administration to a subject as is known in the art. Examples of a unit-dosage form include an ampoule, syringe, and individually packaged tablet and capsule. A unit-dosage form may be administered in fractions or multiples thereof.

The pharmaceutical compositions provided herein may be administered at once, or multiple times at intervals of time. It is understood that the precise dosage and duration of treatment may vary with the age, weight, and condition of the patient being treated, and may be determined empirically using known testing protocols or by extrapolation from in vivo or in vitro test or diagnostic data. It is further understood that for any particular individual, specific dosage regimens can be adjusted over time according to individual need and the professional judgment of the person administering or supervising the administration of the pharmaceutical compositions provided herein.

Exemplary pharmaceutical compositions and components for use therewith are described in U.S. Provisional Application No. 61/088,697, the contents of which are herein incorporated by reference.

A. Oral Administration

The pharmaceutical compositions provided herein may be provided in solid, semisolid, or liquid dosage forms for oral administration. As used herein, oral administration also includes buccal, lingual, and sublingual administration. Suitable oral dosage forms include, but are not limited to, tablets, capsules, pills, troches, lozenges, pastilles, cachets, pellets, medicated chewing gum, granules, bulk powders, effervescent or non-effervescent powders or granules, solutions, emulsions, suspensions (e.g., aqueous or oil suspensions), wafers, sprinkles, elixirs, syrups, bolus, electuaries, or pastes. In addition to the active ingredient(s), the pharmaceutical compositions may contain one or more pharmaceutically acceptable carriers or excipients, including, but not limited to, binders, fillers, diluents, disintegrants, wetting agents, lubricants, glidants, coloring agents, dye-migration inhibitors, preserving agents, sweetening agents, and flavoring agents.

Binders or granulators impart cohesiveness to a tablet to ensure the tablet remaining intact after compression. Suitable binders or granulators include, but are not limited to, starches, such as corn starch, potato starch, and pre-gelatinized starch (e.g., STARCH 1500); gelatin; sugars, such as sucrose, glucose, dextrose, molasses, and lactose; natural and synthetic gums, such as acacia, alginic acid, alginates, extract of Irish moss, panwar gum, Bhatti gum, mucilage of isabgol husks, carboxymethylcellulose, methylcellulose, polyvinylpyrrolidone (PVP), Veegum, larch arabogalactan, powdered tragacanth, and guar gum; celluloses, such as ethyl cellulose, cellulose acetate, carboxymethyl cellulose calcium, sodium carboxymethyl cellulose, methyl cellulose, hydroxyethylcellulose (HEC), hydroxypropylcellulose (HPC), hydroxypropyl methyl cellulose (HPMC); microcrystalline celluloses, such as AVICEL-PH-101, AVICEL-PH-103, AVICEL RC-581, AVICEL-PH-105 (FMC Corp., Marcus Hook, Pa.); and mixtures thereof. Suitable fillers include, but are not limited to, talc, calcium carbonate, microcrystalline cellulose, powdered cellulose, dextrates, kaolin, mannitol, silicic acid, sorbitol, starch, pre-gelatinized starch, and mixtures thereof. In certain embodiments, the binder or filler is present from about 50 to about 99% by weight in the pharmaceutical compositions provided herein.

Suitable diluents include, but are not limited to, dicalcium phosphate, calcium sulfate, calcium carbonate, sodium carbonate, sodium phosphate, lactose, sorbitol, sucrose, inositol, cellulose, kaolin, mannitol, sodium chloride, dry starch, and powdered sugar. Certain diluents, such as mannitol, lactose, sorbitol, sucrose, and inositol, when present in sufficient quantity, can impart properties to some compressed tablets that permit disintegration in the mouth by chewing. Such compressed tablets can be used as chewable tablets.

Suitable disintegrants include, but are not limited to, agar; bentonite; celluloses, such as methylcellulose and carboxymethylcellulose; wood products; natural sponge; cation-exchange resins; alginic acid; gums, such as guar gum and Veegum HV; citrus pulp; cross-linked celluloses, such as croscarmellose; cross-linked polymers, such as crospovidone; cross-linked starches; calcium carbonate; microcrystalline cellulose, such as sodium starch glycolate; polacrilin potassium; starches, such as corn starch, maize starch, potato starch, tapioca starch, and pre-gelatinized starch; clays; aligns; and mixtures thereof. The amount of a disintegrant in the pharmaceutical compositions provided herein varies upon the type of formulation, and is readily discernible to those of ordinary skill in the art. In certain embodiments, the pharmaceutical compositions provided herein contain from about 0.5 to about 15% or from about 1 to about 5% by weight of a disintegrant.

Suitable lubricants include, but are not limited to, calcium stearate; magnesium stearate; mineral oil; light mineral oil; glycerin; sorbitol; mannitol; glycols, such as glycerol behenate and polyethylene glycol (PEG); stearic acid; sodium lauryl sulfate; talc; hydrogenated vegetable oil, including peanut oil, cottonseed oil, sunflower oil, sesame oil, olive oil, corn oil, and soybean oil; zinc stearate; ethyl oleate; ethyl laureate; agar; starch; lycopodium; silica or silica gels, such as AEROSIL® 200 (W. R. Grace Co., Baltimore, Md.) and CAB-0-SM® (Cabot Co. of Boston, Mass.); and mixtures thereof. In certain embodiments, the pharmaceutical compositions provided herein contain about 0.1 to about 5% by weight of a lubricant.

Suitable glidants include, but are not limited to, colloidal silicon dioxide, CAB-0-SIL® (Cabot Co. of Boston, Mass.), and asbestos-free talc. Coloring agents include, but are not limited to, any of the approved, certified, water soluble FD&C dyes, water insoluble FD&C dyes suspended on alumina hydrate, and color lakes, and mixtures thereof. A color lake is the combination by adsorption of a water-soluble dye to a hydrous oxide of a heavy metal, resulting in an insoluble form of the dye. Flavoring agents include, but are not limited to, natural flavors extracted from plants, such as fruits, and synthetic blends of compounds which produce a pleasant taste sensation, such as peppermint and methyl salicylate. Sweetening agents include, but are not limited to, sucrose, lactose, mannitol, syrups, glycerin, and artificial sweeteners, such as saccharin and aspartame. Suitable emulsifying agents include, but are not limited to, gelatin, acacia, tragacanth, bentonite, and surfactants, such as polyoxyethylene sorbitan monooleate (TWEEN® 20), polyoxyethylene sorbitan monooleate 80 (TWEEN® 80), and triethanolamine oleate. Suspending and dispersing agents include, but are not limited to, sodium carboxymethylcellulose, pectin, tragacanth, Veegum, acacia, sodium carbomethylcellulose, hydroxypropyl methylcellulose, and polyvinylpyrrolidone. Preservatives include, but are not limited to, glycerin, methyl and propylparaben, benzoic add, sodium benzoate and alcohol. Wetting agents include, but are not limited to, propylene glycol monostearate, sorbitan monooleate, diethylene glycol monolaurate, and polyoxyethylene lauryl ether. Solvents include, but are not limited to, glycerin, sorbitol, ethyl alcohol, and syrup. Examples of non-aqueous liquids utilized in emulsions include mineral oil and cottonseed oil. Organic acids include, but are not limited to, citric and tartaric acid. Sources of carbon dioxide include, but are not limited to, sodium bicarbonate and sodium carbonate.

It should be understood that many carriers and excipients may serve several functions, even within the same formulation.

The pharmaceutical compositions provided herein may be provided as compressed tablets, tablet triturates, chewable lozenges, rapidly dissolving tablets, multiple compressed tablets, uncoated tablets, enteric coated tablets, sugar-coated tablets, or film-coated tablets. Enteric-coated tablets are compressed tablets coated with substances that resist the action of stomach acid but dissolve or disintegrate in the intestine, thus protecting the active ingredients from the acidic environment of the stomach. Enteric-coatings include, but are not limited to, fatty acids, fats, phenyl salicylate, waxes, shellac, ammoniated shellac, and cellulose acetate phthalates. Sugar-coated tablets are compressed tablets surrounded by a sugar coating, which may be beneficial in covering up objectionable tastes or odors and in protecting the tablets from oxidation. Film-coated tablets are compressed tablets that are covered with a thin layer or film of a water-soluble material. Film coatings include, but are not limited to, hydroxyethylcellulose, sodium carboxymethylcellulose, polyethylene glycol 4000, and cellulose acetate phthalate. Film coating imparts the same general characteristics as sugar coating. Multiple compressed tablets are compressed tablets made by more than one compression cycle, including layered, press-coated, and dry-coated tablets. Tablets may also be coated using microencapsulation to delay disintegration and absorption in the gastrointestinal tract and thereby provide a sustained action over a longer period.

The tablet dosage forms may be prepared from the active ingredient in powdered, crystalline, or granular forms, alone or in combination with a pharmaceutically acceptable vehicle, carrier, diluent, or excipient, or a mixture thereof; including a binder, disintegrant, controlled-release polymer, lubricant, diluent, and/or colorant. Flavoring and sweetening agents are especially useful in the formation of chewable tablets and lozenges. Formulations suitable for topical administration in the mouth include lozenges comprising the active ingredient in a flavored base, usually sucrose and acacia or tragacanth; pastilles comprising the active ingredient in an inert base such as gelatin and glycerin, or sucrose and acacia; and mouthwashes comprising the active ingredient in a suitable liquid carrier.

A tablet may be made by compression or molding, optionally with one or more accessory ingredients. Compressed tablets may be prepared by compressing in a suitable machine a compound provided herein in a free flowing form such as a powder or granules, optionally mixed with a binder (e.g., povidone, gelatin, hydroxypropylmethyl cellulose), lubricant, inert diluent, preservative, disintegrant (e.g., sodium starch glycolate, cross linked povidone, cross linked sodium carboxymethyl cellulose) surface active or dispersing agent. Molded tablets may be made by molding in a suitable machine a mixture of the powdered compound moistened with an inert liquid diluent. The tablets may optionally be coated or scored and may be formulated so as to provide slow or controlled release of the active ingredient therein using, for example, hydroxypropyl methylcellulose in varying proportions to provide the desired release profile. Tablets may optionally be provided with an enteric coating, to provide release in parts of the gut other than the stomach. This can be useful in embodiments where the compounds provided herein are susceptible to acid hydrolysis.

The pharmaceutical compositions provided herein may be provided as soft or hard capsules, which can be made from gelatin, methylcellulose, starch, or calcium alginate. The hard gelatin capsule, also known as a dry-gilled capsule (DFC), consists of two sections, one slipping over the other, thus completely enclosing the active ingredient. The soft elastic capsule (SEC) is a soft, globular shell, such as a gelatin shell, which is plasticized by the addition of glycerin, sorbitol, or a similar polyol. The soft gelatin shells may contain a preservative to prevent the growth of microorganisms. Suitable preservatives are those as described herein, including, but not limited to, methyl- and propyl-parabens, and sorbic acid. The liquid, semisolid, and solid dosage forms provided herein may be encapsulated in a capsule. Suitable liquid and semisolid dosage forms include, but are not limited to, solutions and suspensions in propylene carbonate, vegetable oils, or triglycerides. Capsules containing such solutions can be prepared as described in U.S. Pat. Nos. 4,328,245; 4,409,239; and 4,410,545, The capsules may also be coated as known by those of skill in the art in order to modify or sustain dissolution of the active ingredient. In other embodiments, a hard gelatin capsule contains a mixture of a compound provided herein and an inert solid diluent, e.g., calcium phosphate or kaolin. In other embodiments, a soft gelatin capsule contains a mixture of a compound provided herein and an inert fluidic diluent, e.g., water or an oil medium such as peanut oil, liquid paraffin, or olive oil.

The pharmaceutical compositions provided herein may be provided in liquid and semisolid dosage forms, including, but not limited to, emulsions, solutions, suspensions, elixirs, and syrups. An emulsion is a two-phase system, in which one liquid is dispersed in the form of small globules throughout another liquid, which can be oil-in-water or water-in-oil. Emulsions may include a pharmaceutically acceptable non-aqueous liquid or solvent, emulsifying agent, and preservative.

Suspensions may include a pharmaceutically acceptable suspending agent and preservative. In some embodiments, aqueous suspensions contain an admixture of a compound provided herein and an excipient suitable for the manufacture of aqueous suspensions. Examples of suitable excipients a suspending agent, such as sodium carboxymethylcellulose, methylcellulose, ethylcellulose, hydroxypropylcellulose, hydroxypropyl methylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension may also contain one or more preservatives such as ethyl or n propyl p hydroxy benzoate, one or more coloring agents, one or more flavoring agents and one or more sweetening agents, such as sucrose or saccharin.

Oil suspensions may be formulated by suspending a compound provided herein in a vegetable oil, such as arachid oil, olive oil, sesame oil or coconut oil, or in a mineral oil such as liquid paraffin. Suspensions for oral administration may contain a thickening agent, such as beeswax, hard paraffin or cetyl alcohol, sweetening agent, and/or a flavoring agent. Such compositions may be preserved by the addition of an antioxidant such as ascorbic acid.

Dispersible powders and granules of compounds provided herein are suitable for preparation of an aqueous suspension by the addition of water provide an admixture of a compound provided herein and a dispersing or wetting agent, a suspending agent, and one or more preservatives. Additional excipients, for example sweetening, flavoring and coloring agents, may also be present.

Aqueous alcoholic solutions may include a pharmaceutically acceptable acetal, such as a di(lower alkyl) acetal of a lower alkyl aldehyde, e.g., acetaldehyde diethyl acetal; and a water-miscible solvent having one or more hydroxyl groups, such as propylene glycol and ethanol.

Elixirs are clear, sweetened, and hydroalcoholic solutions. Syrups are concentrated aqueous solutions of a sugar, for example, sucrose, and may also contain a preservative. For a liquid dosage form, for example, a solution in a polyethylene glycol may be diluted with a sufficient quantity of a pharmaceutically acceptable liquid carrier, e.g., water, to be measured conveniently for administration. Syrups and elixirs may be formulated with sweetening agents, such as glycerol, sorbitol or sucrose. Such formulations may also contain a demulcent, a preservative, a flavoring or a coloring agent.

Other useful liquid and semisolid dosage forms include, but are not limited to, those containing the active ingredient(s) provided herein, and a dialkylated mono- or polyalkylene glycol, including, 1,2-dimethoxymethane, diglyme, triglyme, tetraglyme, polyethylene glycol-350-dimethyl ether, polyethylene glycol-550-dimethyl ether, polyethylene glycol-750-dimethyl ether, wherein 350, 550, and 750 refer to the approximate average molecular weight of the polyethylene glycol. These formulations may further comprise one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, bisulfite, sodium metabisulfite, thiodipropionic acid and its esters, and dithiocarbamates.

The pharmaceutical compositions provided herein for oral administration may be also provided in the forms of liposomes, micelles, microspheres, or nanosystems. Micellar dosage forms can be prepared as described in U.S. Pat. No. 6,350,458.

The pharmaceutical compositions provided herein may be provided as noneffervescent or effervescent, granules and powders, to be reconstituted into a liquid dosage form. Pharmaceutically acceptable carriers and excipients used in the non-effervescent granules or powders may include diluents, sweeteners, and wetting agents. Pharmaceutically acceptable carriers and excipients used in the effervescent granules or powders may include organic acids and a source of carbon dioxide.

Coloring and flavoring agents can be used in all of the dosage forms described herein.

As described in greater detail below, the pharmaceutical compositions provided herein may be formulated as immediate or modified release dosage forms, including delayed-, sustained, pulsed-, controlled, targeted-, and programmed-release forms.

The pharmaceutical compositions provided herein may be co-formulated with other active ingredients which do not impair the desired therapeutic action, or with substances that supplement the desired action.

B. Parenteral Administration

The pharmaceutical compositions provided herein may be administered parenterally by injection, infusion, or implantation, for local or systemic administration. Parenteral administration, as used herein, include intravenous, intraarterial, intraperitoneal, intrathecal, intraventricular, intraurethral, intrasternal, intracranial, intramuscular, intrasynovial, and subcutaneous administration.

The pharmaceutical compositions provided herein may be formulated in any dosage forms that are suitable for parenteral administration, including solutions, suspensions, emulsions, micelles, liposomes, microspheres, nanosystems, and solid forms suitable for solutions or suspensions in liquid prior to injection. Such dosage forms can be prepared according to conventional methods known to those skilled in the art of pharmaceutical science (see, Remington: The Science and Practice of Pharmacy, supra).

The pharmaceutical compositions intended for parenteral administration may include one or more pharmaceutically acceptable carriers and excipients, including, but not limited to, aqueous vehicles, water-miscible vehicles, non-aqueous vehicles, bacteriostats, solutes which render the formulation isotonic with the blood of the intended recipient, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, cryoprotectants, lyoprotectants, thickening agents, pH adjusting agents, and inert gases.

Suitable aqueous vehicles include, but are not limited to, water, saline, physiological saline or phosphate buffered saline (PBS), sodium chloride injection, Ringer's injection, isotonic dextrose injection, sterile water injection, and dextrose and lactated Ringer's injection. Non-aqueous vehicles include, but are not limited to, oils including synthetic mono- or diglycerides, fixed oils of vegetable origin, castor oil, corn oil, cottonseed oil, olive oil, peanut oil, peppermint oil, safflower oil, sesame oil, soybean oil, hydrogenated vegetable oils, hydrogenated soybean oil, medium-chain triglycerides of coconut oil, oleic acid, and palm seed oil. Water-miscible vehicles include, but are not limited to, ethanol, 1,3-butanediol, liquid polyethylene glycol (e.g., polyethylene glycol 300 and polyethylene glycol 400), propylene glycol, glycerin, N-methyl-2-pyrrolidone, N,N-dimethylacetamide, and dimethyl sulfoxide.

Suitable antimicrobial agents or preservatives include, but are not limited to, phenols, cresols, mercurials, benzyl alcohol, chlorobutanol, methyl and propyl phydroxyhenzoates, thimerosal, benzalkonium chloride (e.g., benzethonium chloride), methyl- and propyl-parabens, and sorbic acid. Suitable isotonic agents include, but are not limited to, sodium chloride, glycerin, and dextrose. Suitable buffering agents include, but are not limited to, phosphate and citrate. Suitable antioxidants are those as described herein, including bisulfite and sodium metabisulfite. Suitable local anesthetics include, but are not limited to, procaine hydrochloride. Suitable suspending and dispersing agents are those as described herein, including sodium carbomethylcelluose, hydroxypropyl methylcellulose, and polyvinylpyrrolidone. Suitable emulsifying agents include those described herein, including polyoxyethylene sorbitan monolaurate, polyoxyethylene sorbitan monooleate 80, and triethanolamine oleate. Suitable sequestering or chelating agents include, but are not limited to, EDTA. Suitable pH adjusting agents include, but are not limited to, sodium hydroxide, hydrochloric acid, citric acid, and lactic acid. Suitable complexing agents include, but are not limited to, cyclodextrins, including a-cyclodextrin, b-cyclodextrin, hydroxypropyl-b-cyclodextrin, sulfobutylether-b-cyclodextrin, and sulfobutylether 7-b-cyclodextrin (CAPTISOL®, CyDex, Lenexa, Kans.).

The pharmaceutical compositions provided herein may be formulated for single or multiple dosage administration. The single dosage formulations are packaged in an ampoule, a vial, or a syringe. In certain embodiments, the multiple dosage parenteral formulations contain an antimicrobial agent at bacteriostatic or fungistatic concentrations. In certain embodiments, the parenteral formulations provided herein are sterile, as known and practiced in the art.

In one embodiment, the pharmaceutical compositions are provided as ready-to-use sterile solutions. In another embodiment, the pharmaceutical compositions are provided as sterile dry soluble products, including lyophilized powders, hypodermic tablets, or granules, to be reconstituted with a vehicle (such as sterile water for injections) prior to use. In yet another embodiment, the pharmaceutical compositions are provided as ready-to-use sterile suspensions. In yet another embodiment, the pharmaceutical compositions are provided as sterile dry insoluble products to be reconstituted with a vehicle prior to use. In still another embodiment, the pharmaceutical compositions are provided as ready-to-use sterile emulsions.

Formulations suitable for parenteral administration may be administered in a continuous infusion manner, e.g., via an indwelling or external pump or via a hospital bag. The invusions may be done through a Hickman or PICC or any other suitable means of administering a formulation parentally.

The pharmaceutical compositions provided herein may be formulated as immediate or modified release dosage forms, including delayed-, sustained, pulsed-, controlled, targeted-, and programmed-release forms.

The pharmaceutical compositions may be formulated as a suspension, solid, semisolid, or thixotropic liquid, for administration as an implanted depot. In one embodiment, the pharmaceutical compositions provided herein are dispersed in a solid inner matrix, which is surrounded by an outer polymeric membrane that is insoluble in body fluids but allows the active ingredient in the pharmaceutical compositions diffuse through.

Suitable inner matrixes include polymethymethacryate, polybutylmethacrylate, plasticized or unplasticized polyvinylchloride, plasticized nylon, plasticized polyethylene terephthalate, natural rubber, polyisoprene, polyisobutylene, polybutadiene, polyethylene, ethylene-vinyl acetate copolymers, silicone rubbers, polydimethylsiloxanes, silicone carbonate copolymers, hydrophilic polymers, such as hydrogels of esters of acrylic and methacrylic acid, collagen, cross-linked polyvinyl alcohol, and cross-linked partially hydrolyzed polyvinyl acetate.

Suitable outer polymeric membranes include polyethylene, polypropylene, ethylene/propylene copolymers, ethylene/ethyl acrylate copolymers, ethylene/vinyl acetate copolymers, silicone rubbers, polydimethyl siloxanes, neoprene rubber, chlorinated polyethylene, polyvinylchloride, vinyl chloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer.

C. Topical Administration

The pharmaceutical compositions provided herein may be administered topically to the skin, orifices, or mucosa. The topical administration, as used herein, includes (intra)dermal, conjunctival, intracorneal, intraocular, ophthalmic, auricular, transdermal, nasal, vaginal, urethral, respiratory, and rectal administration.

The pharmaceutical compositions provided herein may be formulated in any dosage forms that are suitable for topical administration for local or systemic effect, including emulsions, solutions, suspensions, creams, gels, hydrogels, ointments, dusting powders, dressings, elixirs, lotions, suspensions, tinctures, pastes, foams, films, aerosols, irrigations, sprays, suppositories, bandages, bolus, electuaries, pastes, and dermal patches. The topical formulation of the pharmaceutical compositions provided herein may also comprise liposomes, micelles, microspheres, nanosystems, and mixtures thereof.

Pharmaceutically acceptable carriers and excipients suitable for use in the topical formulations provided herein include, but are not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, penetration enhancers, cryoprotectants, lyoprotectants, thickening agents, and inert gases.

The pharmaceutical compositions may also be administered topically by electroporation, iontophoresis, phonophoresis, sonophoresis, or microneedle or needle-free injection, such as POWDERJECT™ (Chiron Corp., Emeryville, Calif.), and BIOJECT™ (Bioject Medical Technologies Inc., Tualatin, Oreg.).

The pharmaceutical compositions provided herein may be provided in the forms of ointments, creams, and gels. Suitable ointment vehicles include oleaginous or hydrocarbon vehicles, including lard, benzoinated lard, olive oil, cottonseed oil, and other oils; white petrolatum; emulsifiable or absorption vehicles, such as hydrophilic petrolatum, hydroxystearin sulfate, and anhydrous lanolin; water-removable vehicles, such as hydrophilic ointment; water-soluble ointment vehicles, including polyethylene glycols of varying molecular weight; and emulsion vehicles, either water-in-oil (W/O) emulsions or oil-in-water (O/W) emulsions, including cetyl alcohol, glyceryl monostearate, lanolin, and stearic acid (see, Remington: The Science and Practice of Pharmacy, supra). These vehicles are emollient but generally require addition of antioxidants and preservatives.

Suitable cream bases can be oil-in-water or water-in-oil. Cream vehicles may be water-washable, and contain an oil phase, an emulsifier, and an aqueous phase. The oil phase is also called the “internal” phase, which is generally comprised of petrolatum and a fatty alcohol such as cetyl or stearyl alcohol. The aqueous phase usually, although not necessarily, exceeds the oil phase in volume, and generally contains a humectant. The emulsifier in a cream formulation may be a nonionic, anionic, cationic, or amphoteric surfactant.

Gels are semisolid, suspension-type systems. Single-phase gels contain organic macromolecules distributed substantially uniformly throughout a liquid carrier. Suitable gelling agents include crosslinked acrylic acid polymers, such as carbomers, carboxypolyalkylenes, CARBOPOL®; hydrophilic polymers, such as polyethylene oxides, polyoxyethylenepolyoxypropylene copolymers, and polyvinylalcohol; cellulosic polymers, such as hydroxypropyl cellulose, hydroxyethyl cellulose, hydroxypropyl methylcellulose, hydroxypropyl methylcellulose phthalate, and methylcellulose; gums, such as tragacanth and xanthan gum; sodium alginate; and gelatin. In order to prepare a uniform gel, dispersing agents such as alcohol or glycerin can be added, or the gelling agent can be dispersed by trituration, mechanical mixing, and/or stirring.

The pharmaceutical compositions provided herein may be administered rectally, urethrally, vaginally, or perivaginally in the forms of suppositories, pessaries, bougies, poultices or cataplasm, pastes, powders, dressings, creams, plasters, contraceptives, ointments, solutions, emulsions, suspensions, tampons, gels, foams, sprays, or enemas. These dosage forms can be manufactured using conventional processes as described in Remington: The Science and Practice of Pharmacy, supra.

Rectal, urethral, and vaginal suppositories are solid bodies for insertion into body orifices, which are solid at ordinary temperatures but melt or soften at body temperature to release the active ingredient(s) inside the orifices. Pharmaceutically acceptable carriers utilized in rectal and vaginal suppositories include bases or vehicles, such as stiffening agents, which produce a melting point in the proximity of body temperature. Suitable vehicles include, but are not limited to, cocoa butter (theobroma oil), glycerin-gelatin, carbowax (polyoxyethylene glycol), spermaceti, paraffin, white and yellow wax, and appropriate mixtures of mono-, di- and triglycerides of fatty acids, hydro gels, such as polyvinyl alcohol, hydroxyethyl methacrylate, polyacrylic acid; a salicylate, and glycerinated gelatin. Combinations of the various vehicles may be used. Rectal and vaginal suppositories may further comprise antioxidants as described herein, including bisulfite and sodium metabisulfite. Rectal and vaginal suppositories may be prepared by the compressed method or molding. The typical weight of a rectal and vaginal suppository is about 2 to about 3 g.

Formulations suitable for vaginal administration may be presented as pessaries, tampons, creams, gels, pastes, foams or spray formulations containing in addition to the active ingredient such carriers as are known in the art to be appropriate.

The pharmaceutical compositions provided herein may be administered ophthalmically in the forms of solutions, suspensions, ointments, emulsions, gel-forming solutions, powders for solutions, gels, ocular inserts, and implants.

The pharmaceutical compositions provided herein may be administered intranasally or by inhalation to the respiratory tract. The pharmaceutical compositions may be provided in the form of an aerosol or solution for delivery using a pressurized container, pump, spray, atomizer, such as an atomizer using electrohydrodynamics to produce a fine mist, or nebulizer, alone or in combination with a suitable propellant, such as 1,1,1,2-tetrafluoroethane or 1,1,1,2,3,3,3-heptafluoropropane. The pharmaceutical compositions may also be provided as a dry powder for insufflation, alone or in combination with an inert carrier such as lactose or phospholipids; or nasal drops. For intranasal use, the powder may comprise a bioadhesive agent, including chitosan or cyclodextrin.

Solutions or suspensions for use in a pressurized container, pump, spray, atomizer, or nebulizer may be formulated to contain ethanol, aqueous ethanol, or a suitable alternative agent for dispersing, solubilizing, or extending release of the active ingredient provided herein, a propellant as solvent; and/or a surfactant, such as sorbitan trioleate, oleic acid, or an oligolactic acid.

The pharmaceutical compositions provided herein may be micronized to a size suitable for delivery by inhalation, such as about 50 micrometers or less, or about 10 micrometers or less. Particles of such sizes may be prepared using a comminuting method known to those skilled in the art, such as spiral jet milling, fluid bed jet milling, supercritical fluid processing to form nanoparticles, high pressure homogenization, or spray drying.

Capsules, blisters, and cartridges for use in an inhaler or insufflator may be formulated to contain a powder mix of the pharmaceutical compositions provided herein; a suitable powder base, such as lactose or starch; and a performance modifier, such as 1-leucine, mannitol, or magnesium stearate. The lactose may be anhydrous or in the form of monohydrates. Other suitable excipients or carriers include dextran, glucose, maltose, sorbitol, xylitol, fructose, sucrose, and trehalose. The pharmaceutical compositions provided herein for inhaled/intranasal administration may further comprise a suitable flavor, such as menthol and levomenthol, or sweeteners, such as saccharin or saccharin sodium.

The pharmaceutical compositions provided herein for topical administration may be formulated to be immediate release or modified release, including delayed-, sustained-, pulsed-, controlled-, targeted, and programmed release.

In some embodiments, a “pill and patch” strategy may be taken, in which a fraction of the daily dose is provided topically (e.g., transdermally) to generate basal systemic levels, and an oral therapy further provided.

D. Modified Release

The pharmaceutical compositions provided herein may be formulated as a modified release dosage form. As used herein, the term “modified release” refers to a dosage form in which the rate or place of release of the active ingredient(s) is different from that of an immediate dosage form When administered by the same route. Modified release dosage forms include delayed-, extended-, prolonged-, sustained-, pulsatile-, controlled-, accelerated- and fast-, targeted-, programmed-release, and gastric retention dosage forms, The pharmaceutical compositions in modified release dosage forms can be prepared using a variety of modified release devices and methods known to those skilled in the art, including, but not limited to, matrix controlled release devices, osmotic controlled release devices, multiparticulate controlled release devices, ion-exchange resins, enteric coatings, multilayered coatings, microparticles, microspheres, liposomes, and combinations thereof. The release rate of the active ingredient(s) can also be modified by varying the particle sizes and polymorphorism of the active ingredient(s).

Additionally, ion exchange materials can be used to prepare immobilized, adsorbed co-crystals and thus effect controlled delivery of the drug. Examples of specific anion exchanges include, but are not limited to, Duolite A568 and Duolite AP143 (Rohm & Haas, Spring House, Pa., USA).

Examples of modified release include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,639,480; 5,733,566; 5,739,108; 5,891,474; 5,922,356; 5,972,891; 5,980,945; 5,993,855; 6,045,830; 6,087,324; 6,113,943; 6,197,350; 6,248,363; 6,264,970; 6,267,981; 6,365,185; 6,376,461; 6,419,961; 6,589,548; 6,613,358; and 6,699,500.

1. Matrix Controlled Release Devices

The pharmaceutical compositions provided herein in a modified release dosage form may be fabricated using a matrix controlled release device known to those skilled in the art (see, Takada et al in “Encyclopedia of Controlled Drug Delivery,” Vol. 2, Mathiowitz Ed., Wiley, 1999) (incorporated herein by reference).

In one embodiment, the pharmaceutical compositions provided herein is formulated in a modified release dosage form using an erodible matrix device, which is water-swellable, erodible, or soluble polymers, including synthetic polymers, and naturally occurring polymers and derivatives, such as polysaccharides and proteins.

Materials useful in forming an erodible matrix include, but are not limited to, chitin, chitosan, dextran, and pullulan; gum agar, gum arabic, gum karaya, locust bean gum, gum tragacanth, carrageenans, gum ghatti, guar gum, xanthan gum, and scleroglucan; starches, such as dextrin and maltodextrin; hydrophilic colloids, such as pectin; phosphatides, such as lecithin; alginates; propylene glycol alginate; gelatin; collagen; and cellulosics, such as ethyl cellulose (EC), methylethyl cellulose (MEC), carboxymethyl cellulose (CMC), CMEC, hydroxyethyl cellulose (HEC), hydroxypropyl cellulose (HPC), cellulose acetate (CA), cellulose propionate (CP), cellulose butyrate (CB), cellulose acetate butyrate (CAB), CAP, CAT, by methyl cellulose (HPMC), HPMCP, HPMCAS, hydroxypropyl methyl cellulose acetate trimellitate (HPMCAT), and ethylhydroxy ethylcellulose (EHEC); polyvinyl pyrrolidone; polyvinyl alcohol; polyvinyl acetate; glycerol fatty acid esters; polyacrylamide; polyacrylic acid; copolymers of ethacrylic acid or methacrylic acid (EUDRAGIT®, Rohm America, Inc., Piscataway, N.J.); poly(2-hydroxyethyl-methacrylate); polylactides; copolymers of L-glutamic acid and ethyl-L-glutamate; degradable lactic acid-glycolic acid copolymers; poly-D-(7)-3-hydroxybutyric acid; and other acrylic acid derivatives, such as homopolymers and copolymers of butylmethacrylate, methylmethacrylate, ethylmethacrylate, ethylacrylate, (2-dimethylaminoethyl)methacrylate, and (trimethylaminoethyl)methacrylate chloride.

In certain embodiments, the pharmaceutical compositions are formulated with a non-erodible matrix device. The active ingredient(s) is dissolved or dispersed in an inert matrix and is released primarily by diffusion through the inert matrix once administered. Materials suitable for use as a non-erodible matrix device include, but are not limited to, insoluble plastics, such as polyethylene, polypropylene, polyisoprene, polyisobutylene, polybutadiene, polymethylmethacrylate, polybutylmethactylate, chlorinated polyethylene, polyvinylchloride, methyl acrylate-methyl methacrylate copolymers, ethylene-vinyl acetate copolymers, ethylene/propylene copolymers, ethylene/ethyl acrylate copolymers, vinyl chloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, polyvinyl chloride, plasticized nylon, plasticized polyethylene terephthalate, natural rubber, silicone rubbers, polydimethylsiloxanes, silicone carbonate copolymers; hydrophilic polymers, such as ethyl cellulose, cellulose acetate, crospovidone, and cross-linked partially hydrolyzed polyvinyl acetate; and fatty compounds, such as carnauba wax, microcrystalline wax, and triglycerides.

In a matrix controlled release system, the desired release kinetics can be controlled, for example, via the polymer type employed, the polymer viscosity, the particle sizes of the polymer and/or the active ingredient(s), the ratio of the active ingredient(s) versus the polymer, and other excipients or carriers in the compositions.

The pharmaceutical compositions provided herein in a modified release dosage form may be prepared by methods known to those skilled in the art, including direct compression, dry or wet granulation followed by compression, or melt-granulation followed by compression.

2. Osmotic Controlled Release Devices

The pharmaceutical compositions provided herein in a modified release dosage form may be fabricated using an osmotic controlled release device, including one-chamber system, two-chamber system, asymmetric membrane technology (AMT), and extruding core system (ECS). In general, such devices have at least two components: (a) the core which contains the active ingredient(s); and (b) a semipermeable membrane with at least one delivery port, which encapsulates the core. The semipermeable membrane controls the influx of water to the core from an aqueous environment of use so as to cause drug release by extrusion through the delivery port(s).

In addition to the active ingredient(s), the core of the osmotic device optionally includes an osmotic agent, which creates a driving force for transport of water from the environment of use into the core of the device. One class of osmotic agents water-swellable hydrophilic polymers, which are also referred to as “osmopolymers” and “hydrogels,” including, but not limited to, hydrophilic vinyl and acrylic polymers, polysaccharides such as calcium alginate, polyethylene oxide (PEO), polyethylene glycol (PEG), polypropylene glycol (PPG), poly(2-hydroxyethyl methacrylate), poly(acrylic) acid, poly(methacrylic) add, polyvinylpyrrolidone (PVP), crosslinked PVP, polyvinyl alcohol (PV A), PV A/PVP copolymers, PV A/PVP copolymers with hydrophobic monomers such as methyl methacrylate and vinyl acetate, hydrophilic polyurethanes containing large PEO blocks, sodium croscarmellose, carrageenan, hydroxyethyl cellulose (HEC), hydroxypropyl cellulose (HPC), hydroxypropyl methyl cellulose (HPMC), carboxymethyl cellulose (CMC) and carboxyethyl, cellulose (CEC), sodium alginate, polycarbophil, gelatin, xanthan gum, and sodium starch glycolate.

The other class of osmotic agents includes osmogens, which are capable of imbibing water to affect an osmotic pressure gradient across the barrier of the surrounding coating. Suitable osmogens include, but are not limited to, inorganic salts, such as magnesium sulfate, magnesium chloride, calcium chloride, sodium chloride, lithium chloride, potassium sulfate, potassium phosphates, sodium carbonate, sodium sulfite, lithium sulfate, potassium chloride, and sodium sulfate; sugars, such as dextrose, fructose, glucose, inositol, lactose, maltose, mannitol, raffinose, sorbitol, sucrose, trehalose, and xylitol; organic acids, such as ascorbic acid, benzoic acid, fumaric acid, citric acid, maleic acid, sebacic acid, sorbic acid, adipic acid, edetic acid, glutamic acid, p-toluenesulfonic acid, succinic acid, and tartaric acid; urea; and mixtures thereof.

Osmotic agents of different dissolution rates may be employed to influence how rapidly the active ingredient(s) is initially delivered from the dosage form. For example, amorphous sugars, such as MANNOGEM™ EZ (SPI Pharma, Lewes, Del.) can be used to provide faster delivery during the first couple of hours to promptly produce the desired therapeutic effect, and gradually and continually release of the remaining amount to maintain the desired level of therapeutic or prophylactic effect over an extended period of time. In this case, the active ingredient(s) is released at such a rate to replace the amount of the active ingredient metabolized and excreted.

The core may also include a wide variety of other excipients and carriers as described herein to enhance the performance of the dosage form or to promote stability or processing.

Materials useful in forming the semipermeable membrane include various grades of acrylics, vinyls, ethers, polyamides, polyesters, and cellulosic derivatives that are water-permeable and water-insoluble at physiologically relevant pHs, or are susceptible to being rendered water-insoluble by chemical alteration, such as crosslinking. Examples of suitable polymers useful in forming the coating, include plasticized, unplasticized, and reinforced cellulose acetate (CA), cellulose diacetate, cellulose triacetate, CA propionate, cellulose nitrate, cellulose acetate butyrate (CAB), CA ethyl carbamate, CAP, CA methyl carbamate, CA succinate, cellulose acetate trimellitate (CAT), CA dimethylaminoacetate, CA ethyl carbonate, CA chloroacetate, CA ethyl oxalate, CA methyl sulfonate, CA butyl sulfonate, CA p-toluene sulfonate, agar acetate, amylase triacetate, beta glucan acetate, beta glucan triacetate, acetaldehyde dimethyl acetate, triacetate of locust bean gum, hydroxylated ethylene-vinylacetate, EC, PEG-, PPG, PEG/PPG copolymers, PVP, HEC, HPC, CMC, CMEC, HPMC, HPMCP, HPMCAS, HPMCAT, poly(acrylic) acids and esters, poly-(methacrylic) acids and esters, and copolymers thereof, starch, dextran, dextrin, chitosan, collagen, gelatin, polyalkenes, polyethers, polysulfones, polyethersulfones, polystyrenes, polyvinyl halides, polyvinyl esters and ethers, natural waxes, and synthetic waxes.

Semipermeable membrane may also be a hydrophobic macroporous membrane, wherein the pores are substantially filled with a gas and are not wetted by the aqueous medium but are permeable to water vapor, as disclosed in U.S. Pat. No. 5,798,119. Such hydrophobic but water-vapor permeable membrane are typically composed of hydrophobic polymers such as polyalkenes, polyethylene, polypropylene, polytetrafluoroethylene, polyacrylic acid derivatives, polyethers, polysulfones, polyethersulfones, polystyrenes, polyvinyl halides, polyvinylidene fluoride, polyvinyl esters and ethers, natural waxes, and synthetic waxes.

The delivery port(s) on the semipermeable membrane may be formed post-coating by mechanical or laser drilling. Delivery port(s) may also be formed in situ by erosion of a plug of water-soluble material or by rupture of a thinner portion of the membrane over an indentation in the core. In addition, delivery ports may be formed during coating process, as in the case of asymmetric membrane coatings as described in U.S. Pat. Nos. 5,612,059 and 5,698,220.

The total amount of the active ingredient(s) released and the release rate can substantially by modulated via the thickness and porosity of the semipermeable membrane, the composition of the core, and the number, size, and position of the delivery ports.

The pharmaceutical compositions in an osmotic controlled-release dosage form may further comprise additional conventional excipients or carriers as described herein to promote performance or processing of the formulation.

The osmotic controlled-release dosage forms can be prepared according to conventional methods and techniques known to those skilled in the art (see, Remington: The Science and Practice of Pharmacy, supra; Santus and Baker, J. Controlled Release 1995, 35, 1-21; Verma et al., Drug Development and Industrial Pharmacy 2000, 26, 695-708; Verma et at., J. Controlled Release 2002, 79, 7-27).

In certain embodiments, the pharmaceutical compositions provided herein are formulated as an AMT controlled-release dosage form, which comprises an asymmetric osmotic membrane that coats a core comprising the active ingredients) and other pharmaceutically acceptable excipients or carriers. See, U.S. Pat. No. 5,612,059 and WO 2002/17918. The AMT controlled-release dosage forms can be prepared according to conventional methods and techniques known to those skilled in the art, including direct compression, dry granulation, wet granulation, or dip-coating method.

In certain embodiments, the pharmaceutical compositions provided herein are formulated as ESC controlled-release dosage form, which comprises an osmotic membrane that coats a core comprising the active ingredient(s), a hydroxylethyl cellulose, and other pharmaceutically acceptable excipients or carriers.

One example of a suitable osmotic drug delivery system is referred to as OROS (Alza Corporation, Mountain View, Calif. USA). Various aspects of the technology are disclosed in U.S. Pat. Nos. 6,375, 978; 6,368,626; 6,342,249; 6,333,050; 6,287,295; 6,283,953; 6,270,787; 6,245,357; and 6,132,420; each of which is incorporated herein by reference in its entirety. Specific adaptations of OROS that can be used to administer compounds and compositions provided herein include, but are not limited to, the OROS, Push-Pull, Delayed Push-Pull, MultiLayer Push-Pull, and Push-Stick Systems available from Alza Corporation. Additional OROS systems that can be used for the controlled oral delivery of compounds and compositions provided herein include OROS-CT and L-OROS, also available from Alza Corporation.

OROS oral dosage forms may be made by compressing a drug powder into a hard tablet, coating the tablet with cellulose derivatives to form a semi-permeable membrane, and then drilling an orifice in the coating (e.g., with a laser). For further details, see Kim, Cherug-ju, Controlled Release Dosage Form Design, 231-238 (Technomic Publishing, Lancaster, Pa.: 2000). One feature of such dosage forms is that the delivery rate of the drug is not influenced by physiological or experimental conditions. Even a drug with a pH-dependent solubility can be delivered at a constant rate regardless of the pH of the delivery medium.

In one embodiment, a dosage form is provided in which a wall is formed defining a cavity, the wall having an exit orifice formed or formable therein and at least a portion of the wall being semipermeable; an expandable layer located within the cavity remote from the exit orifice and in fluid communication with the semipermeable portion of the wall; a dry or substantially dry state drug layer located within the cavity adjacent to the exit orifice and in direct or indirect contacting relationship with the expandable layer; and a flow-promoting layer interposed between the inner surface of the wall and at least the external surface of the drug layer located within the cavity, wherein the drug layer includes a crystalline form of a compound provided herein. For further details, see U.S. Pat. No. 6,368,626, the entirety of which is incorporated herein by reference.

In another embodiment, a dosage is provided in which a wall is formed defining a cavity, the wall having an exit orifice formed or formable therein and at least a portion of the wall being semipermeable; an expandable layer located within the cavity remote from the exit orifice and in fluid communication with the semipermeable portion of the wall; a drug layer located within the cavity adjacent the exit orifice and in direct or indirect contacting relationship with the expandable layer, the drug layer comprising a liquid, active agent formulation absorbed in porous particles, the porous particles being adapted to resist compaction forces sufficient to form a compacted drug layer without significant exudation of the liquid, active agent formulation, the dosage form optionally having a placebo layer between the exit orifice and the drug layer, wherein the active agent includes a crystalline form of a compound provided herein. For further details, see U.S. Pat. No. 6,342,249, the entirety of which is incorporated herein by reference.

3. Multiparticulate Controlled Release Devices

The pharmaceutical compositions provided herein in a modified release dosage form may be fabricated a multiparticulate controlled release device, which comprises a multiplicity of particles, granules, or pellets, from about 10 μm to about 3 mm, about 50 μm to about 2.5 mm, or from about 100 μm to about 1 mm in diameter. Such multiparticulates may be made by the processes know to those skilled in the art, including wet-and dry-granulation, extrusion/spheronization, roller-compaction, melt-congealing, and by spray-coating seed cores. See, for example, Multiparticulate Oral Drug Delivery; Marcel Dekker: 1994; and Pharmaceutical Pelletization Technology; Marcel Dekker: 1989.

Other excipients or carriers as described herein may be blended with the pharmaceutical compositions to aid in processing and forming the multiparticulates. The resulting particles may themselves constitute the multiparticulate device or may be coated by various film-forming materials, such as enteric polymers, water-swellable, and water-soluble polymers. The multiparticulates can be further processed as a capsule or a tablet.

4. Targeted Delivery

The pharmaceutical compositions provided herein may also be formulated to be targeted to a particular tissue, receptor, or other area of the body of the subject to be treated, including liposome-, resealed erythrocyte-, and antibody-based delivery systems. Examples include, but are not limited to, those described in U.S. Pat. Nos. 6,316,652; 6,274,552; 6,271,359; 6,253,872; 6,139,865; 6,131,570; 6,120,751; 6,071,495; 6,060,082; 6,048,736; 6,039,975; 6,004,534; 5,985,307; 5,972,366; 5,900,252; 5,840,674; 5,759,542; and 5,709,874.

c. Methods of Use

In one embodiment, provided herein is a method of treating, preventing, or ameliorating one or more symptoms of a condition, disorder, or disease associated with impaired glucose tolerance, a metabolic syndrome, or a glucagon receptor, comprising administering to a subject having or being suspected to have such a condition, disorder, or disease, a therapeutically effective amount of a compound provided herein, e.g., a compound of Formula I or II, or a pharmaceutically acceptable salt, solvate, or prodrug thereof; or a pharmaceutical composition thereof. In one embodiment, the subject is a mammal. In another embodiment, the subject is a human.

In another embodiment, provided herein is a method of treating, preventing, or ameliorating one or more symptoms of a condition, disorder, or disease responsive to a decrease in the hepatic glucose production or in the blood glucose level of a subject, comprising administering to the subject having or being suspected to have such a condition, disorder, or disease, a therapeutically effective amount of a compound provided herein, e.g., a compound of Formula I or II, or a pharmaceutically acceptable salt, solvate, or prodrug thereof; or a pharmaceutical composition thereof. In one embodiment, the subject is a mammal. In another embodiment, the subject is a human.

The conditions and diseases treatable with the methods provided herein include, but are not limited to, type 1 diabetes, type 2 diabetes, gestational diabetes, ketoacidosis, nonketotic hyperosmolar coma (nonketotic hyperglycemia), impaired glucose tolerance (IGT), insulin resistance syndromes, syndrome X, low HDL levels, high LDL levels, hyperglycemia, hyperinsulinemia, hyperlipidemia, hypertriglyceridemia, hyperlipoproteinemia, hypercholesterolemia, dyslipidemia, arteriosclerosis, atherosclerosis, glucagonomas, acute pancreatitis, cardiovascular diseases, hypertension, cardiac hypertrophy, gastrointestinal disorders, obesity, vascular restenosis, pancreatitis, neurodegenerative disease, retinopathy, nephropathy, neuropathy, accelerated gluconeogenesis, excessive (greater than normal levels) hepatic glucose output, and lipid disorders.

Provided herein are also methods of delaying the time to onset or reducing the risk of the development or progression of a disease or condition responsive to decreased hepatic glucose production or responsive to lowered blood glucose levels.

Depending on the condition, disorder, or disease to be treated and the subject's condition, a compound provided herein may be administered by oral, parenteral (e.g., intramuscular, intraperitoneal, intravenous or intraarterial (e.g., via catheter), ICV, intracistemal injection or infusion, subcutaneous injection, or implant), inhalation, nasal, vaginal, rectal, sublingual, and/or topical (e.g., transdermal or local) routes of administration, and may be formulated alone or together in suitable dosage unit with a pharmaceutically acceptable vehicle, carrier, diluent, excipient, or a mixture thereof, appropriate for each route of administration.

The dose may be in the form of one, two, three, four, five, six, or more sub-doses that are administered at appropriate intervals per day. The dose or sub-doses can be administered in the form of dosage units containing from about from about 0.01 to 2500 mg, from about 0.1 mg to about 1,000 mg, from about 1 mg to about 1000 mg, from about 1 mg to about 500 mg, from about 0.1 mg to about 500 mg, from about 0.1 mg to about 100 mg, from about 0.5 mg about to about 100 mg, from about 1 mg to about 100 mg, from about 10 mg to about 1000 mg, from about 10 mg to about 500 mg, or from about 10 mg to about 100 mg of active ingredient(s) per dosage unit. For example, the dose or subdoses can be administered in the form of dosage units containing about 10 mg, about 25 mg, about 50 mg, about 75 mg, about 100 mg, about 250 mg, about 300 mg, about 400 mg, about 500 mg, about 600 mg, about 700 mg, about 800 mg, about 900 mg, or about 1000 mg. If the condition of the patient requires, the dose can, by way of alternative, be administered as a continuous infusion.

In certain embodiments, an appropriate dosage level is about 0.01 to about 100 mg per kg patient body weight per day (mg/kg per day), about 0.01 to about 50 mg/kg per day, about 0.01 to about 25 mg/kg per day, or about 0.05 to about 10 mg/kg per day, which may be administered in single or multiple doses. A suitable dosage level may be about 0.01 to about 100 mg/kg per day, about 0.05 to about 50 mg/kg per day, or about 0.1 to about 10 mg/kg per day. Within this range, the dosage may be about 0.01 to about 0.1, about 0.1 to about 1.0, about 1.0 to about 10, or about 10 to about 50 mg/kg per day.

For oral administration, the pharmaceutical compositions can be provided in the form of tablets containing 1.0 to 1,000 mg of the active ingredient, for example, about 1, about 5, about 10, about 15, about 20, about 25, about 50, about 75, about 100, about 150, about 200, about 250, about 300, about 400, about 500, about 600, about 750, about 800, about 900, and about 1,000 mg of the active ingredient for the symptomatic adjustment of the dosage to the patient to be treated. The compositions may be administered on a regimen of 1 to 4 times per day, including once, twice, three times, and four times per day. In various embodiments, the compositions may be administered before a meal, after a meal, in the morning hours, after awakening, in the evening hours, and/or at bedtime.

It will be understood, however, that the specific dose level, frequency, and timing of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

In still another embodiment, provided herein is a method of modulating the biological activity of a glucagon receptor, comprising contacting the receptor with one or more of the compounds provided herein, e.g., a compound of Formulas I or II, including a single enantiomer, a mixture of enantiomers, or a mixture of diastereomers thereof, or a pharmaceutically acceptable salt, solvate, or prodrug thereof; or a pharmaceutical composition thereof. In one embodiment, the glucagon receptor is expressed by a cell.

The compounds provided herein may also be combined or used in combination with each other or other therapeutic agents useful in the treatment, prevention, or amelioration of one or more symptoms of the conditions, disorders, or diseases for which the compounds provided herein are useful. As used herein, the term “in combination” includes the use of more than one therapeutic agents. However, the use of the term “in combination” does not restrict the order in which therapeutic agents are administered to a subject with a condition, disorder, or disorder. A first therapeutic agent (e.g., a therapeutic agent such as a compound provided herein) can be administered prior to (e.g., 5 min, 15 min, 30 min, 45 min, 1 hr, 2 hrs, 4 hrs, 6 hrs, 12 hrs, 24 hrs, 48 hrs, 72 hrs, 96 hrs, 1 wk, 2 wks, 3 wks, 4 wks, 5 wks, 6 wks, 8 wks, or 12 wks before), concomitantly with, or subsequent to (e.g., 5 min, 15 min, 30 min, 45 min, 1 hr, 2 hrs, 4 hrs, 6 hrs, 12 hrs, 24 hrs, 48 hrs, 72 hrs, 96 hrs, 1 wk, 2 wks, 3 wks, 4 wks, 5 wks, 6 wks, 8 wks, or 12 wks after) the administration of a second therapeutic agent to a subject to be treated.

When a compound provided herein is used contemporaneously with one or more additional therapeutic agents, a pharmaceutical composition containing such other agents in addition to the compound provided herein may be utilized, but is not required. Accordingly, the pharmaceutical compositions provided herein include those that also contain one or more other therapeutic agents, in addition to a compound provided herein.

In one embodiment, the other therapeutic agent is an antidiabetic agent. Suitable antidiabetic agents include, but are not limited to, insulin sensitizers, biguanides (e.g., metformin), PPAR agonists (e.g., triglitazone, pioglitazone, and rosiglitazone), insulin and insulin mimetics, somatostatin, α-glucosidase inhibitors (e.g., voglibose, miglitol, and acarbose), dipeptidyl peptidase-4 inhibitors, SGLT-2 inhibitors, liver X receptor modulators, insulin secretagogues (e.g., acetohexamide, carbutamide, chlorpropamide, glibornuride, gliclazide, glimerpiride, glipizide, gliquidine, glisoxepid, glyburide, glyhexamide, glypinamide, phenbutamide, sulfonylureas, tolazamide, tolbutamide, tolcyclamide, nateglinide, and repaglinide), other glucagon receptor antagonists, GLP-1, GLS-1 mimetics exenatide, liraglutide, DPPIV inhibitors), GLP-1 receptor agonists, GIP, GIP mimetics, GIP receptor agonists, PACAP, PACAP mimetics, PACAP receptor 3 agonists, cholesterol lowering agents, HMG-CoA reductase inhibitors (e.g., statins, such as lovastatin, simvastatin, pravastatin, fluvastatin, atorvastatin, itavastatin, rivastatin, NK-104 (a.k.a. itavastatin, nisvastatin, and nisbastatin), and ZD-4522 (also known as rosuvastatin, atavastatin, and visastatin)), a cholesterol absorption inhibitor (e.g., ezetimibe), sequestrants, nicotinyl alcohol, nicotinic acid and salts thereof, PPAR α agonists, PPAR α/γ dual agonists, inhibitors of cholesterol absorption, acyl CoA:cholesterol acyltransferase inhibitors, anti-oxidants, PPAR δ agonists, antiobesity compounds, ileal bile acid transporter inhibitors, anti-inflammatory agents, and protein tyrosine phosphatase-1B (PTP-1B) inhibitors.

The dosages given will depend on absorption, inactivation and excretion rates of the drug as well as other factors known to those of skill in the art. It is to be noted that dosage values will also vary with the severity of the condition to be alleviated. It is to be further understood that for any particular subject, specific dosage regimens and schedules should be adjusted over time according to the individual need and the professional judgment of the person administering or supervising the administration of the compositions.

The weight ratio of a compound provided herein to the second active ingredient depends upon the effective dose of each ingredient. Generally, an effective dose of each will be used. Thus, for example, when a compound provided herein is combined with a PPAR agonist the weight ratio of the compound provided herein to the PPAR agonist will generally range from about 1000:1 to about 1:1000 or about 200:1 to about 1:200. Combinations of a compound provided herein and other active ingredients will generally also be within the aforementioned range, but in each case, an effective dose of each active ingredient should be used.

### SYNTHESIS OF COMPOUNDS

Compounds of Formula I and can be prepared according to the methodology outlined in the following general synthetic schemes or with modifications of these schemes that will be evident to persons skilled in the art, or by other methods readily known to those of skill in the art.

The compounds of structure 4 can be generated using the synthetic strategy shown in Scheme 1. Briefly, a carboxylic acid of structure 1 is coupled with a compound of structure 2 to afford a coupled compound of structure 3. Then, reaction of the acid of structure 3 with taurine generates the final compound of structure 4.

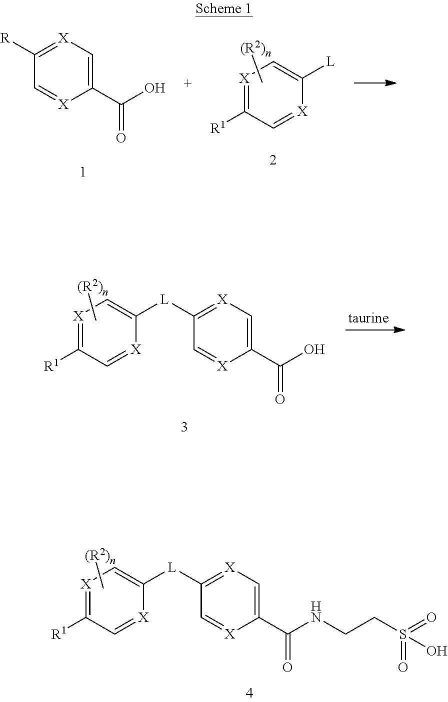

An alternative route for the synthesis of the final compound of structure 4 is shown in Scheme 2. The sulfonic acid moiety of a compound of structure 6 is introduced first by amide coupling between the carboxylic acid of structure 5 and taurine. Linking reactions between a compound of structure 6 and a compound of structure 7 provides the compound of structure 4.

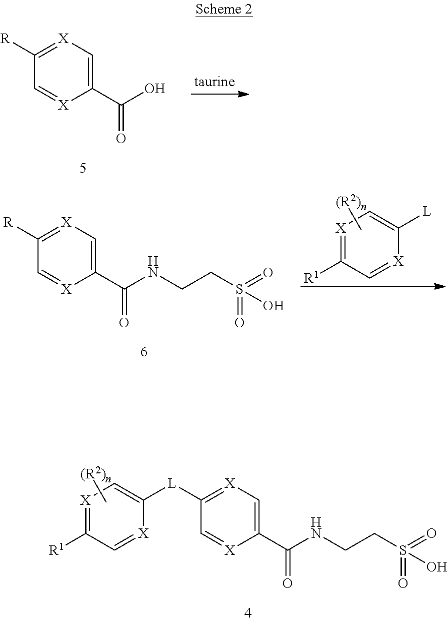

The following examples are provided so that this disclosure can be more fully understood. They should not be construed as limiting the disclosure in any way.

### EXAMPLES

### Example 1

### (±)-2-(4-(2-(4-(tert-Butyl)phenyl)-2-(5-(4-isobutylphenyl)-1H-benzo[d]imidazol-2-yl)ethyl)benzamido)ethane-1-sulfonic acid (Compound 101)

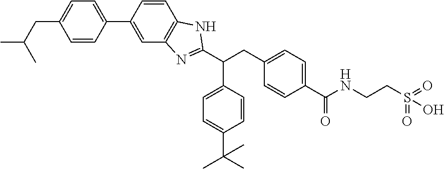 [SMILES: CC(C)Cc1ccc(-c2ccc3[nH]c(C(Cc4ccc(C(=O)NCCS(=O)(=O)O)cc4)c4ccc(C(C)(C)C)cc4)nc3c2)cc1]

Compound 101 was prepared as a colorless solid according to the general procedure described in Scheme 2 from commercially available starting material by the specific reaction sequence in Scheme 3. Alkylation of acetic acid 8 with benzyl bromide 9 in basic condition affords product 10. Amide formation between the acid 10 and aniline 11 followed by intramolecular condensation under basic condition gives the benzimidazole compound 12. Treatment of compound 12 with taurine generates sulfonic acid intermediate 13 that is coupled with boronic acid 14 catalyzed by a palladium ion to complete Compound 101 synthesis. Calculated elemental analysis of C₃₈H₄₃N₃O₄S•0.5H₂O: C, 70.56; H, 6.86; and N, 6.50; and found: C, 70.55; H, 6.89; and N, 6.73.

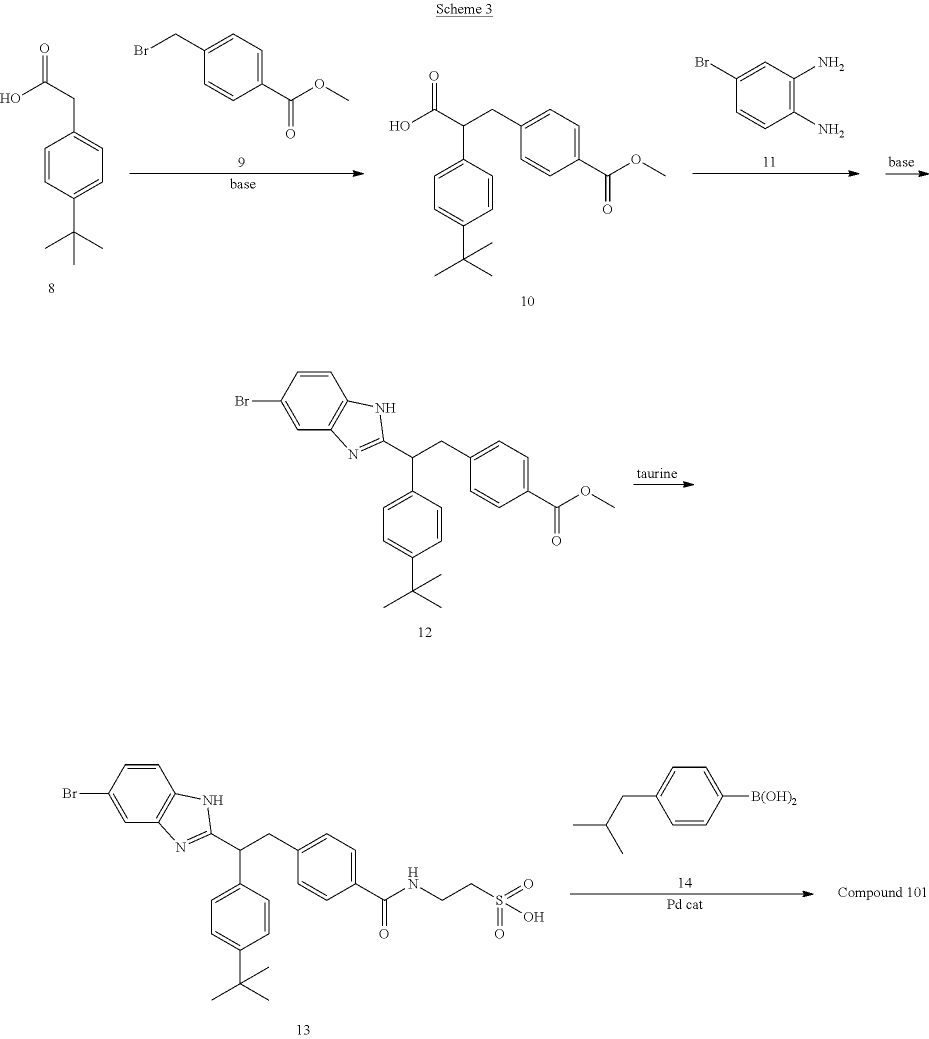

### Example 2

### 2-(4((2-(4-(tert-Butyl)phenyl)-4-(4-(trifluoromethoxy)phenyl)methyl)benzamido)ethane-1-sulfonic acid (Compound 102)

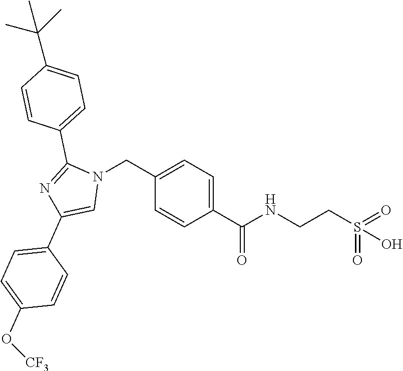 [SMILES: CC(C)(C)c1ccc(-c2nc(-c3ccc(OC(F)(F)F)cc3)cn2Cc2ccc(C(=O)NCCS(=O)(=O)O)cc2)cc1]

Compound 102 was prepared as a white solid according to the general procedure from commercially available starting material that is similar to the synthetic sequence in Scheme 3. Calculated elemental analysis of C₃₀H₃₀F₃N₃O₅S•1.0H₂O•0.2TFA: C, 56.83; H, 5.05; and N, 6.54; and found: C, 56.78; H, 5.01; and N, 6.35.

### Example 3

### 2-(4((5-(tert-Butyl)-2-(2′,4′-dichloro-[1,1′-biphenyl]-4-yl)-1H-benzo[d]imidazol-1-yl)methyl)benzamido)ethane-1-sulfonic acid (Compound 103)

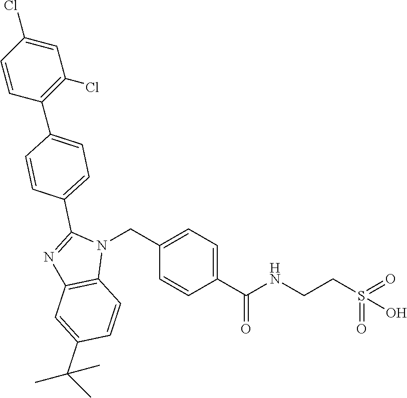 [SMILES: CC(C)(C)c1ccc2c(c1)nc(-c1ccc(-c3ccc(Cl)cc3Cl)cc1)n2Cc1ccc(C(=O)NCCS(=O)(=O)O)cc1]

Compound 103 was prepared as a colorless solid according to the general procedure from commercially available starting material that is similar to the synthetic sequence in Scheme 3, Calculated elemental analysis of C₃₃H₃₁N₃O₄Cl₂S•1.6H₂O: C, 59.57; H, 5.18; and N, 6.31; and found: C, 59.54; H, 4.95; and N, 6.48.

### Example 4

### 2-(4-((4-(tert-Butyl)-2-(2′,4′-dichloro-[1,1′-biphenyl]-4-yl)-1H-benzo[d]imidazol-1-yl)methyl)benzamido)ethane-1-sulfonic acid (Compound 104)

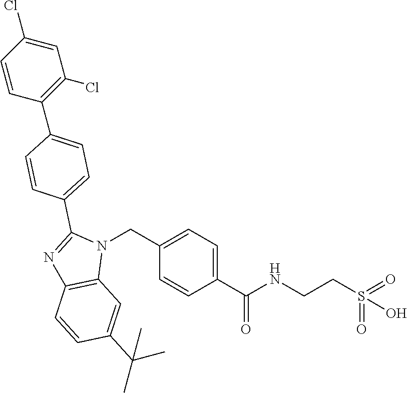 [SMILES: CC(C)(C)c1ccc2nc(-c3ccc(-c4ccc(Cl)cc4Cl)cc3)n(Cc3ccc(C(=O)NCCS(=O)(=O)O)cc3)c2c1]

Compound 104 was prepared as a yellow solid according to the general procedure from commercially available starting material that is similar to the synthetic sequence in Scheme 3. Calculated elemental analysis of C₃₃H₃₁N₃O₄Cl₂S•1.1H₂O•0.7MeOH: C, 59.63; H, 5.35; and N, 6.19; and found: C, 59.97; H, 5.74; and N, 6.45.

### Example 5

### 2-(4-((2-(4-(Benzofuran-2-yl)phenyl)-5-(tert-butyl)-1H-benzo[d]imidazol-1-yl)methyl)benzamido)ethane-1-sulfonic acid (Compound 105)

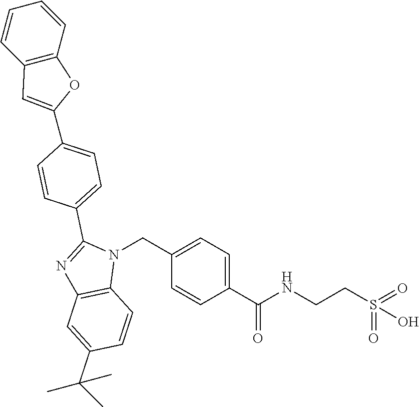 [SMILES: CC(C)(C)c1ccc2c(c1)nc(-c1ccc(-c3cc4ccccc4o3)cc1)n2Cc1ccc(C(=O)NCCS(=O)(=O)O)cc1]

Compound 105 was prepared as a colorless solid according to the general procedure from commercially available starting material that is similar to the synthetic sequence in Scheme 3. Calculated elemental analysis of C₃₅H₃₃N₃O₅S•1.5MeOH: C, 66.85; H, 5.99; and N, 6.41; and found: C, 66.88; H, 6.30; and N, 6.80.

### Example 6

### 2-(4-((2-(4-(Benzofuran-2-yl)phenyl)-4-(tert-butyl)-1H-benzo[d]imidazol-1-yl)methyl)benzamido)ethane-1-sulfonic acid (Compound 106)

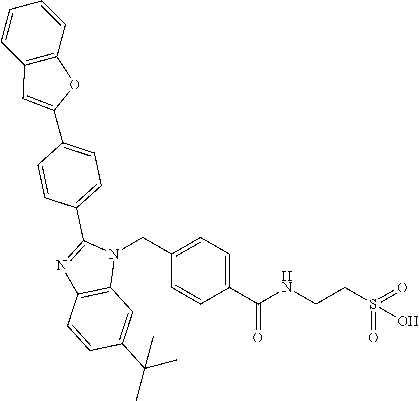 [SMILES: CC(C)(C)c1ccc2nc(-c3ccc(-c4cc5ccccc5o4)cc3)n(Cc3ccc(C(=O)NCCS(=O)(=O)O)cc3)c2c1]

Compound 106 was prepared as a colorless solid according to the general procedure from commercially available starting material that is similar to the synthetic sequence in Scheme 3. Calculated elemental analysis of C₃₅H₃₃N₃O₅S•0.9MeOH: C, 67.74; H, 5.80; and N, 6.60; and found: C, 67.84; H, 6.18; and N, 6.94.

### Example 7

### 2-(4-((2-(3,3-Dimethylbutyl)-4-phenyl-1H-imidazol-1-yl)methyl)benzamido)ethane-1-sulfonic acid (Compound 107)

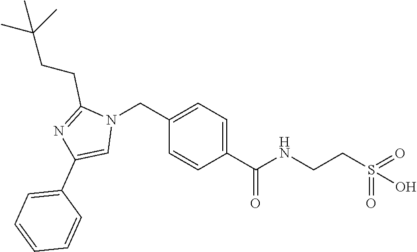 [SMILES: CC(C)(C)CCc1nc(-c2ccccc2)cn1Cc1ccc(C(=O)NCCS(=O)(=O)O)cc1]

Compound 107 was prepared as a colorless solid according to the general procedure from commercially available starting material that is similar to the synthetic sequence in Scheme 3. Molecular weight of C₂₅H₃₁N₃O₄S is 469.61; (MH⁺): calculated 470.2 and observed 470.4.

### Example 8

### (±)-2-(4-(2-(4′-(tert-Butyl)-[1,1′-biphenyl]-4-yl)-2-(5-mesityl-1H-benzo[d]imidazol-2-yl)ethyl)benzamido)ethane -1-sulfonic acid (Compound 108)

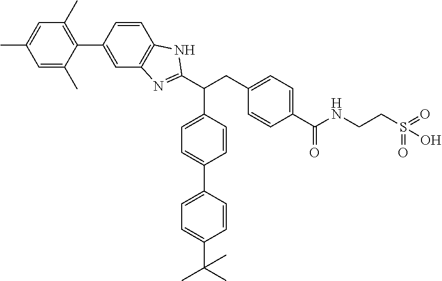 [SMILES: Cc1cc(C)c(-c2ccc3[nH]c(C(Cc4ccc(C(=O)NCCS(=O)(=O)O)cc4)c4ccc(-c5ccc(C(C)(C)C)cc5)cc4)nc3c2)c(C)c1]

Compound 108 was prepared as a colorless solid according to the general procedure from commercially available starting material that is similar to the synthetic sequence in Scheme 3. Molecular weight of C₄₃H₄₅N₃O₄S is 699.31; m/z (MH⁺): calculated 700.3 and observed 700.4.

### Example 9

### Sodium (±)-2-(4-(2-(4′-(tert-butyl)-[1,1′-biphenyl]-4-yl)-2-(5-(4-(tert-butyl)cyclohex-1-en-1-yl)-1H-benzo[d]imidazol-2-ypethyl)benzamido)ethane-1-sulfonate (Compound 109)

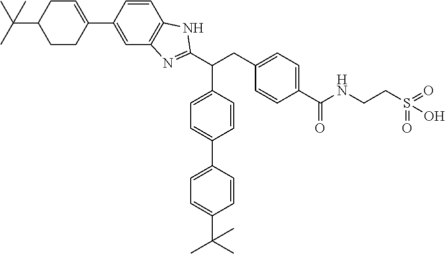 [SMILES: CC(C)(C)c1ccc(-c2ccc(C(Cc3ccc(C(=O)NCCS(=O)(=O)O)cc3)c3nc4cc(C5=CCC(C(C)(C)C)CC5)ccc4[nH]3)cc2)cc1]

Compound 109 was prepared as a colorless solid according to the general procedure from commercially available starting material that is similar to the synthetic sequence in Scheme 3. Molecular weight of C₄₄H₅₁N₃O₄S is 717.36; m/z (MH⁺): calculated 718.4 and observed 718.9.

### Example 10

### (±)-2-(4-(2-(4′-(tert-Butyl)-[1,1′-biphenyl]-4-yl)-2-(5-(4,4-dimethylcyclohex-1-en-1-yl)-1H-benzo[d]imidazol-2-yl)ethyl)benzamido)ethane-1-sulfonic acid (Compound 110)

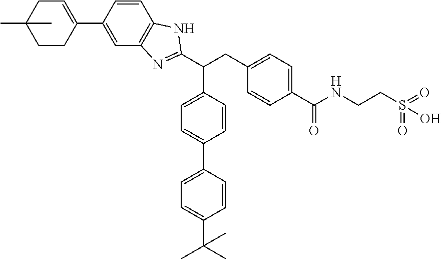

Compound 110 was prepared as a colorless solid according to the general procedure from commercially available starting material that is similar to the synthetic sequence in Scheme 3. Molecular weight of C₄₂H₄₇N₃O₄S is 689.91; m/z (MH⁺): calculated 690.3 and observed 691.1.

### Example 11

### (±)-2-(4-(2-(4′-(tert-Butyl)-[1,1′-biphenyl]-4-yl)-2-(5-(1r,4r)-4-(tert-butyl)cyclohexyl-1H-benzo[d]imidazol-2-yl)ethyl)benzamido)ethane-1-sulfonic acid (Compound 111)

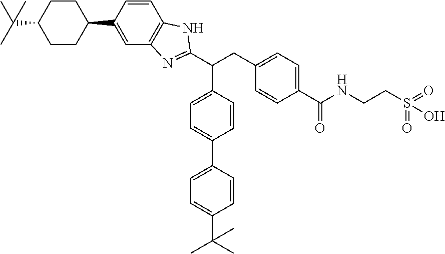 [SMILES: CC(C)(C)c1ccc(-c2ccc(C(Cc3ccc(C(=O)NCCS(=O)(=O)O)cc3)c3nc4cc([C@H]5CC[C@H](C(C)(C)C)CC5)ccc4[nH]3)cc2)cc1]

Compound 111 was prepared as a colorless solid according to the general procedure from commercially available starting material that is similar to the synthetic sequence in Scheme 3. Molecular weight of C₄₄H₅₃N₃O₄S is 719.90; m/z (MH⁺): calculated 720.4 and observed 720.6.

### Example 12

### (±)-2-(4-(2-(4′-(tert-Butyl)-[1,1′-biphenyl]-4-yl)-2-(5-((1s,4s)-4-(tert-butyl)cyclohexyl)-1H-benzo[d]imidazol-2-yl)ethyl)benzamido)ethane-1-sulfonic acid (Compound 112)

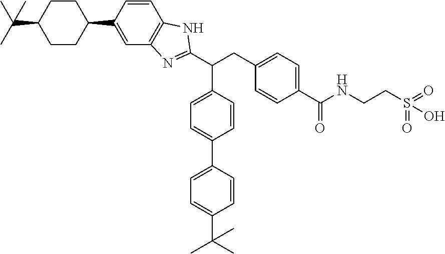 [SMILES: CC(C)(C)c1ccc(-c2ccc(C(Cc3ccc(C(=O)NCCS(=O)(=O)O)cc3)c3nc4cc([C@H]5CC[C@@H](C(C)(C)C)CC5)ccc4[nH]3)cc2)cc1]

Compound 112 was prepared as a fluffy solid according to the general procedure from commercially available starting material that is similar to the synthetic sequence in Scheme 3. Molecular weight of C₄₄H₅₃N₃O₄S is 719.90; m/z (MH⁺): calculated 720.4 and observed 720.6.

### Example 13

### (±)-2-(4-(2-(4′-(tert-Butyl)-[1,1′-biphenyl]-4-yl)-2-(5-(4,4-dimethylcyclohexyl)-1H-benzo[d]imidazol-2-yl)ethyl)benzamido)ethane-1-sulfonic acid (Compound 113)

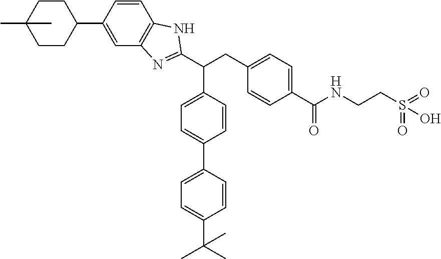

Compound 113 was prepared as a fluffy solid according to the general procedure from commercially available starting material that is similar to the synthetic sequence in Scheme 3. Molecular weight of C₄₄H₅₃N₃O₄S is 691.93; m/z (MH⁺): calculated 692.3 and observed 692.2.

### Example 14

### 2-(4-(1R,2S)-1-(4-Chlorophenyl)-1-(7-fluoro-5-methyl-1H-indol-3-yl)pentan-2-yl)benzamido)ethane -1-sulfonic acid (Compound 114)

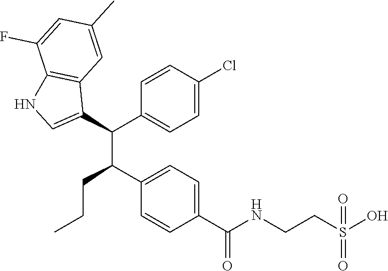 [SMILES: CCC[C@H](c1ccc(C(=O)NCCS(=O)(=O)O)cc1)[C@H](c1ccc(Cl)cc1)c1c[nH]c2c(F)cc(C)cc12]

Compound 114 can be prepared as a solid according to the general procedure from commercially available starting material.

### Example 15

### (R)-2-(4-(1-((4′-(tert-Butyl)-2,6-dimethyl-[1,1′-biphenyl]-4-yl)oxy)-4,4,4-trifluorobutyl)benzamido)ethane -1-sulfonic acid (Compound 115)

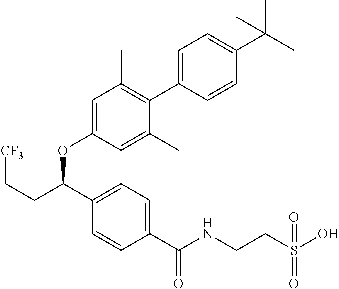 [SMILES: Cc1cc(O[C@H](CCC(F)(F)F)c2ccc(C(=O)NCCS(=O)(=O)O)cc2)cc(C)c1-c1ccc(C(C)(C)C)cc1]

Compound 115 can be prepared as a solid according to the general procedure from commercially available starting material.

### Example 16

### (S)-2-(4-(1-((4′-(tert-Butyl)-2,6-dimethyl-[1,1′-biphenyl]-4-yl)oxy)-4,4,4-trifluorobutyl)benzamido)ethane -1-sulfonic acid (Compound 116)

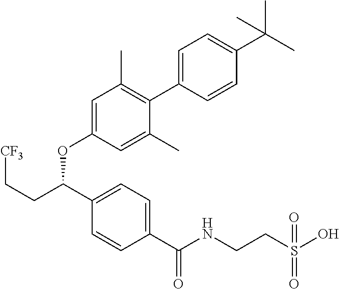 [SMILES: Cc1cc(O[C@@H](CCC(F)(F)F)c2ccc(C(=O)NCCS(=O)(=O)O)cc2)cc(C)c1-c1ccc(C(C)(C)C)cc1]

Compound 116 can be prepared as a solid according to the general procedure from commercially available starting material.

### Example 17

### (R)-2-(4-(1-(3,5-Dimethyl-4-(4-(trifluoromethyl)-1H-pyrazol-1-yl)phenoxy)butyl)benzamido)ethane -1-sulfonic acid (Compound 117)

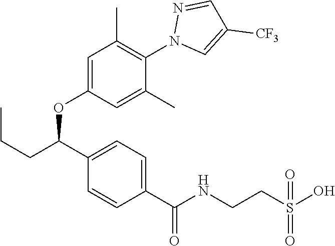 [SMILES: CCC[C@@H](Oc1cc(C)c(-n2cc(C(F)(F)F)cn2)c(C)c1)c1ccc(C(=O)NCCS(=O)(=O)O)cc1]

Compound 117 can be prepared as a solid according to the general procedure from commercially available starting material.

### Example 18

### (S)-2-(4-(1-(3,5-Dimethyl-4-(4-(trifluoromethyl)-1H-pyrazol-1-yl)phenoxy)butyl)benzamido)ethane -1-sulfonic acid (Compound 118)

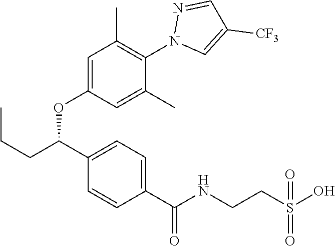 [SMILES: CCC[C@H](Oc1cc(C)c(-n2cc(C(F)(F)F)cn2)c(C)c1)c1ccc(C(=O)NCCS(=O)(=O)O)cc1]

Compound 118 can be prepared as a solid according to the general procedure from commercially available starting material.

### Example 19

### 2-(5-(2-(((2-Chloro-2′,4′,6′-trimethyl-[1,1′-biphenyl]-4-yl)amino)methyl)-5-(trifluoromethyl)phenyl)picolinamido)ethane -1-sulfonic acid (Compound 119)

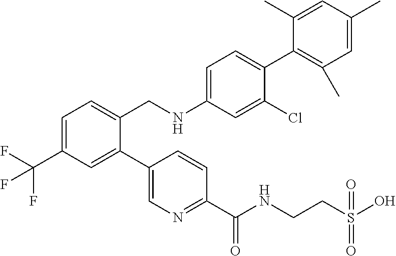 [SMILES: Cc1cc(C)c(-c2ccc(NCc3ccc(C(F)(F)F)cc3-c3ccc(C(=O)NCCS(=O)(=O)O)nc3)cc2Cl)c(C)c1]

Compound 119 can be prepared as a solid according to the general procedure from commercially available starting material.

### Example 20

### 2-(2′(((4′-(tert-Butyl)-[1,1′-biphenyl]-4-yl)amino)methyl)-5′-chloro-[1,1′-biphenyl]-4-carboxamido)ethane -1-sulfonic acid (Compound 120)

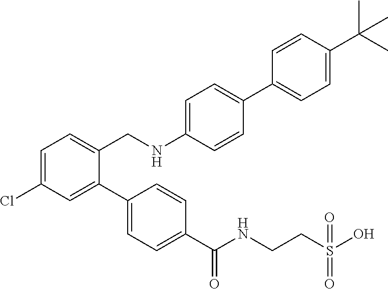 [SMILES: CC(C)(C)c1ccc(-c2ccc(NCc3ccc(Cl)cc3-c3ccc(C(=O)NCCS(=O)(=O)O)cc3)cc2)cc1]

Compound 120 can be prepared as a solid according to the general procedure from commercially available starting material.

### Example 21

### (±)-2-(4-(((1-(4-Chlorophenyl)-3-methyl-1H-pyrazol-4-yl)(cyclohexyl)methyl)amino)benzamido)ethane -1-sulfonic acid (Compound 121)

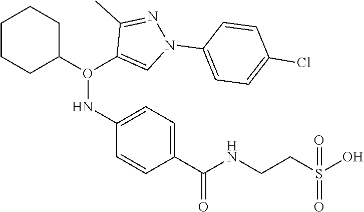

Compound 121 can be prepared as a solid according to the general procedure from commercially available starting material.

### Example 22

### (S)-2-(5-(1-(3-(3,5-Dichlorophenyl)-5-(6-methoxynaphthalen-2-yl)-1H-pyrazol-1-yl)ethyl)thiophene -2-carboxamido)ethane-1-sulfonic acid (Compound 122)

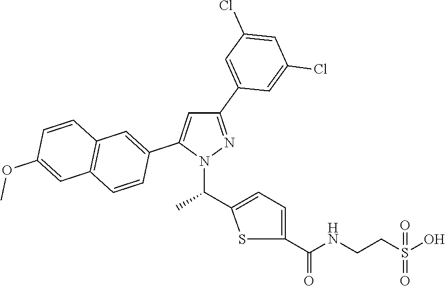 [SMILES: COc1ccc2cc(-c3cc(-c4cc(Cl)cc(Cl)c4)nn3[C@@H](C)c3ccc(C(=O)NCCS(=O)(=O)O)s3)ccc2c1]

Compound 122 can be prepared as a solid according to the general procedure from commercially available starting material.

### Example 23

### (S)-2-(5-(1-((4′-(tert-Butyl)-2,6-dimethyl-[1,1′-biphenyl]-4-yl)oxy)-4,4,4-trifluorobutyl)thiophene -2-carboxamido)ethane-1-sulfonic acid (Compound 123)

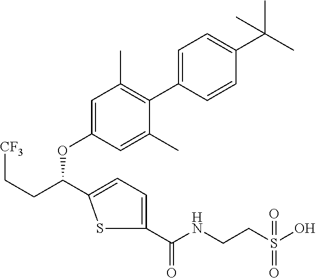 [SMILES: Cc1cc(O[C@@H](CCC(F)(F)F)c2ccc(C(=O)NCCS(=O)(=O)O)s2)cc(C)c1-c1ccc(C(C)(C)C)cc1]

Compound 123 can be prepared as a solid according to the general procedure from commercially available starting material.

### Example 24

### 2-(5-((1R, 2S)-1-(4-Chlorophenyl)-1-(7-fluoro-5-methyl-1H-indol-3-yl)pentan-2-yl)thiophene -2-carboxamido)ethane-1-sulfonic acid (Compound 124)

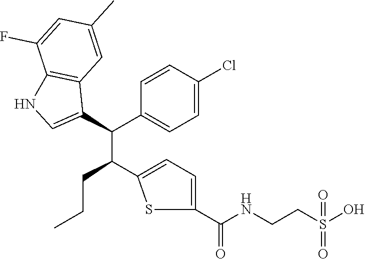 [SMILES: CCC[C@H](c1ccc(C(=O)NCCS(=O)(=O)O)s1)[C@H](c1ccc(Cl)cc1)c1c[nH]c2c(F)cc(C)cc12]

Compound 124 can be prepared as a solid according to the general procedure from commercially available starting material.

### Example 25

### (S)-2-(5-(1-(3,5-Dimethyl-4-(4-(trifluoromethyl)-1H-pyrazol-1-yl)phenoxy)butyl)thiophene -2-carboxamido)ethane-1-sulfonic acid (Compound 125)

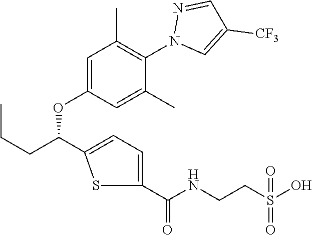 [SMILES: CCC[C@H](Oc1cc(C)c(-n2cc(C(F)(F)F)cn2)c(C)c1)c1ccc(C(=O)NCCS(=O)(=O)O)s1]

Compound 125 can be prepared as a solid according to the general procedure from commercially available starting material.

The examples set forth above are provided to provide those of ordinary skill in the art with a complete disclosure and description of how to make and use the claimed embodiments, and are not intended to limit the scope of what is disclosed herein. Modifications that are obvious to persons of skill in the art are intended to be within the scope of the following claims. All publications, patents, and patent applications cited in this specification are incorporated herein by reference in their entireties as if each such publication, patent or patent application were specifically and individually indicated to be incorporated herein by reference.

1. A compound of Formula I: 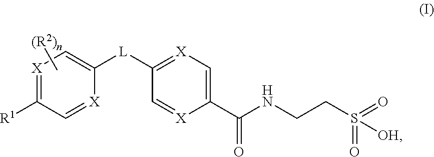
wherein:
R¹ is selected from the group consisting of hydrogen, halogen, an optionally substituted C₁₋₆-alkyl, an optionally substituted C₁₋₆-heteroalkyl, an optionally substituted C₂₋₆-alkenyl, an optionally substituted C₁₋₆-heteroalkenyl, an optionally substituted aryl, and an optionally substituted heteroaryl;
R² is selected from the group consisting of hydrogen, halogen, and an optionally substituted C₁₋₆-alkyl;
R³ is selected from the group consisting of hydrogen, an optionally substituted C₁₋₆-alkyl, and an optionally substituted C₁₋₆-heteroalkyl;
X is independently CH or N;
L is selected from a group consisting of —OCHR³—, —SCHR³—, —NR³CHR³—, —CHR¹CHR³—, —CHR³O—, —CHR³S—, —CHR³NR³—, -arylCHR³—, -arylO—, -heteroarylCHR³—, -heteroarylCHR³NH—, -heteroarylCHR³O—, -heteroarylO—, —CHR³aryl-, —CHR³heteroaryl-, —OCH₂CHR³—, —NHCHR³aryl-, —OCHR³aryl-, and —NHCHR³heteroaryl-, where the aryl and the heteroaryl are independently optionally substituted;
n is 1, 2, or 3;
or a pharmaceutically acceptable salt, solvate, or prodrug thereof.

2. A compound of Formula II: 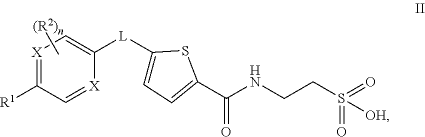
wherein:
R¹ is selected from the group consisting of hydrogen, halogen, an optionally substituted C₁₋₆-alkyl, an optionally substituted C₁₋₆-heteroalkyl, an optionally substituted C₂₋₆-alkenyl, an optionally substituted C₁₋₆-heteroalkenyl, an optionally substituted aryl, and an optionally substituted heteroaryl;
R² is selected from the group consisting of hydrogen, halogen, and an optionally substituted C₁₋₆-alkyl;
R³ is selected from the group consisting of hydrogen, an optionally substituted C₁₋₆alkyl, and an optionally substituted C₁₋₆-heteroalkyl;
X is independently CH or N;
L is selected from a group consisting of —OCHR³—, —SCHR³—, —NR³CHR³—, —CHR¹CHR³—, —CHR³O—, —CHR³S—, —CHR³NR³—, -arylCHR³—, -arylO—, —-heteroarylCHR³—, -heteroarylCHR³NH—, -heteroarylCHR³O—, -heteroarylO—, —CHR³aryl-, —CHR³heteroaryl-, —OCH₂CHR³—, —NHCHR³aryl-, —OCHR³aryl-, and —NHCHR³heteroaryl-, where the aryl and the heteroaryl are independently optionally substituted;
n is 1, 2, or 3;
or a pharmaceutically acceptable salt, solvate, or prodrug thereof.

3. The compound of claim 1, wherein:
R² is H or Cl or CH₃;
R³ is selected from a group of hydrogen, CH₃, CH₂CH₃, CH₂CH₂CH₃, and CH₂CH₂CF₃; X is CH;
L is —OCHR³—, —SCHR³—, —NR³CHR³—, —CHR¹CHR³—, —CHR³O—, —CHR³S—, —CHR³NR³—, -heteroarylCHR³—, -heteroarylO—, —OCH₂CHR³—, —NHCHR³aryl-, and —OCHR³aryl-, where the aryl and the heteroaryl are independently optionally substituted.

4. The compound of claim 3, wherein L is —OCHR³—, —CHR¹CHR³—, and -heteroarylCHR³—, where the heteroaryl is substituted with an aryl optionally substituted with F, Cl, CH₃, OCH₃, and CF₃.

5. A compound selected from Formulae (101) to (125): 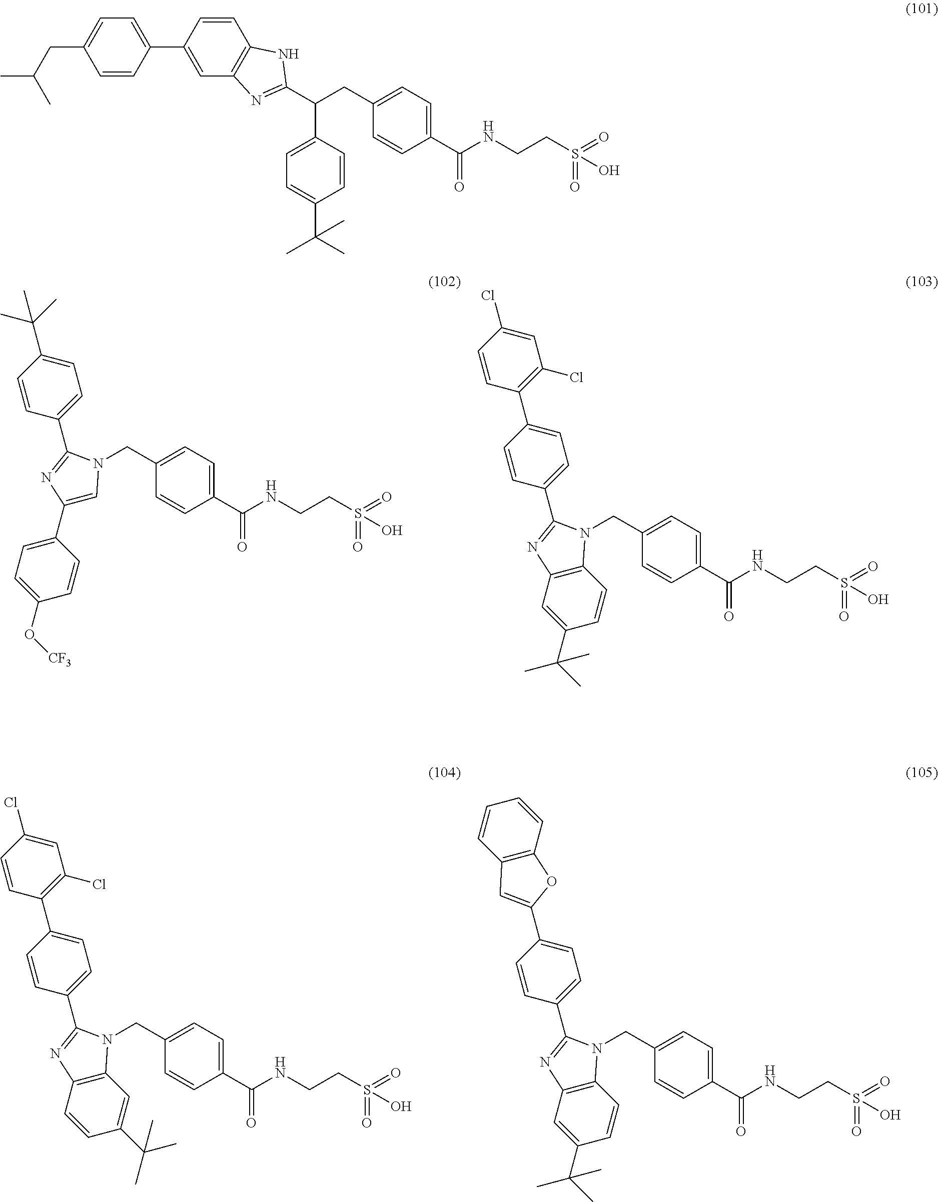 [SMILES: CC(C)Cc1ccc(-c2ccc3[nH]c(C(Cc4ccc(C(=O)NCCS(=O)(=O)O)cc4)c4ccc(C(C)(C)C)cc4)nc3c2)cc1 ; CC(C)(C)c1ccc(-c2nc(-c3ccc(OC(F)(F)F)cc3)cn2Cc2ccc(C(=O)NCCS(=O)(=O)O)cc2)cc1 ; CC(C)(C)c1ccc2c(c1)nc(-c1ccc(-c3ccc(Cl)cc3Cl)cc1)n2Cc1ccc(C(=O)NCCS(=O)(=O)O)cc1 ; CC(C)(C)c1ccc2nc(-c3ccc(-c4ccc(Cl)cc4Cl)cc3)n(Cc3ccc(C(=O)NCCS(=O)(=O)O)cc3)c2c1 ; CC(C)(C)c1ccc2c(c1)nc(-c1ccc(-c3cc4ccccc4o3)cc1)n2Cc1ccc(C(=O)NCCS(=O)(=O)O)cc1] 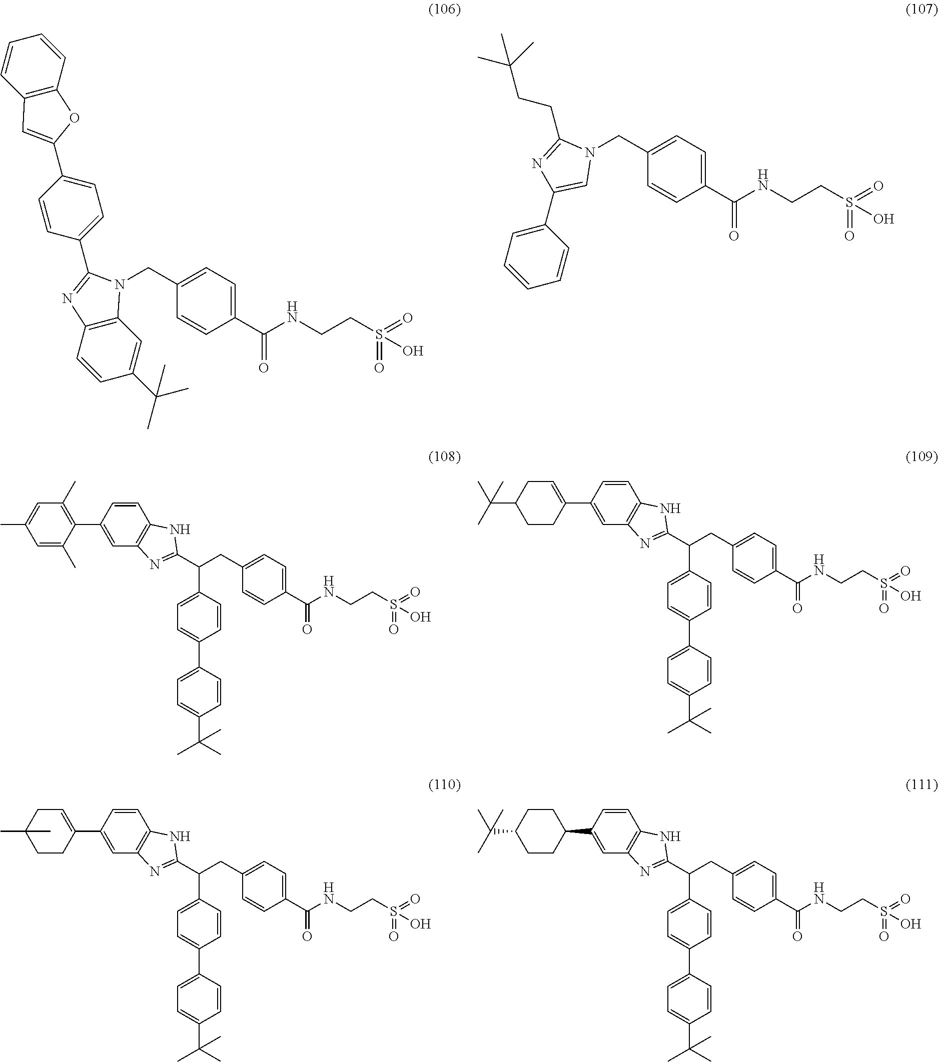 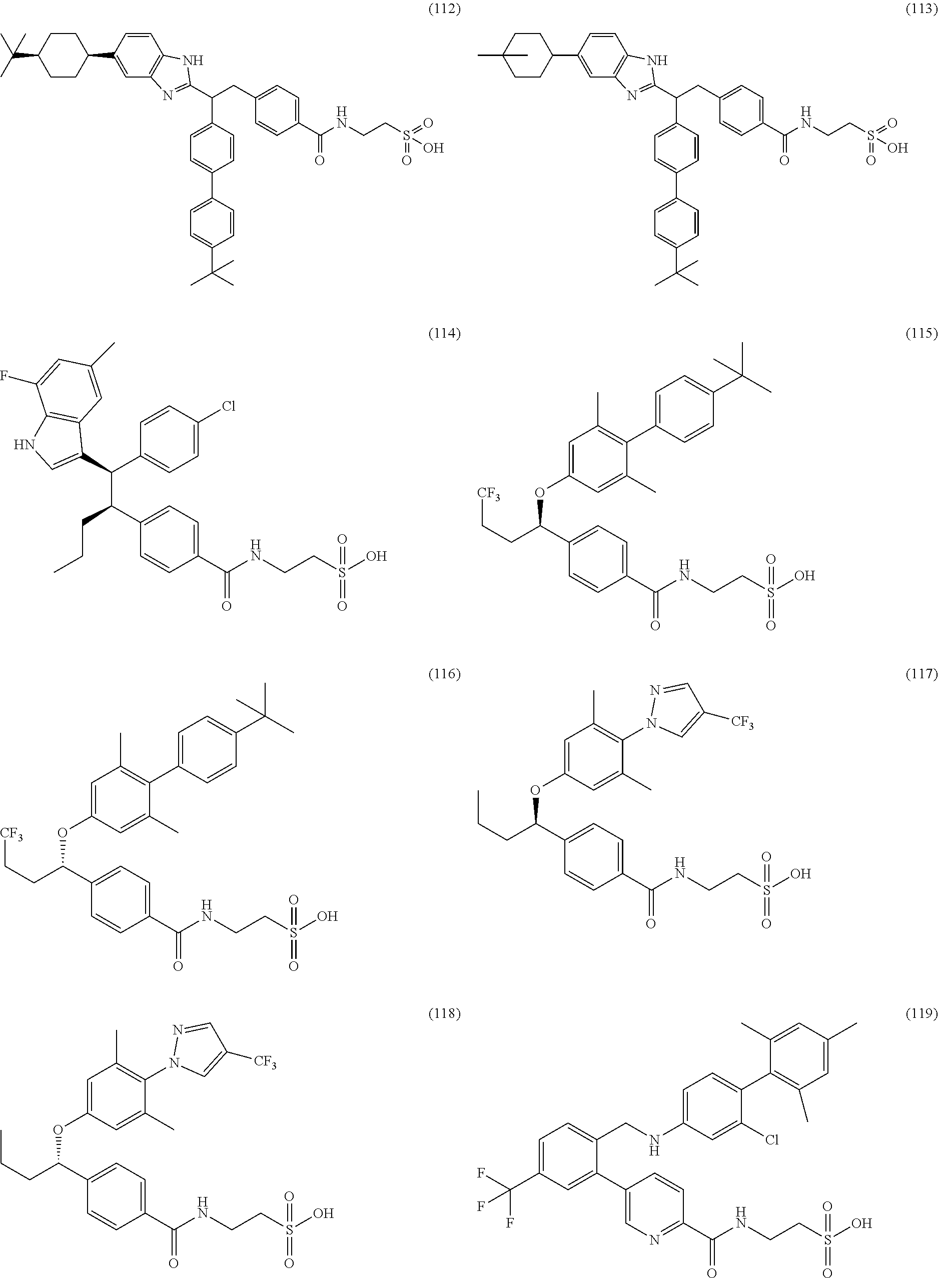 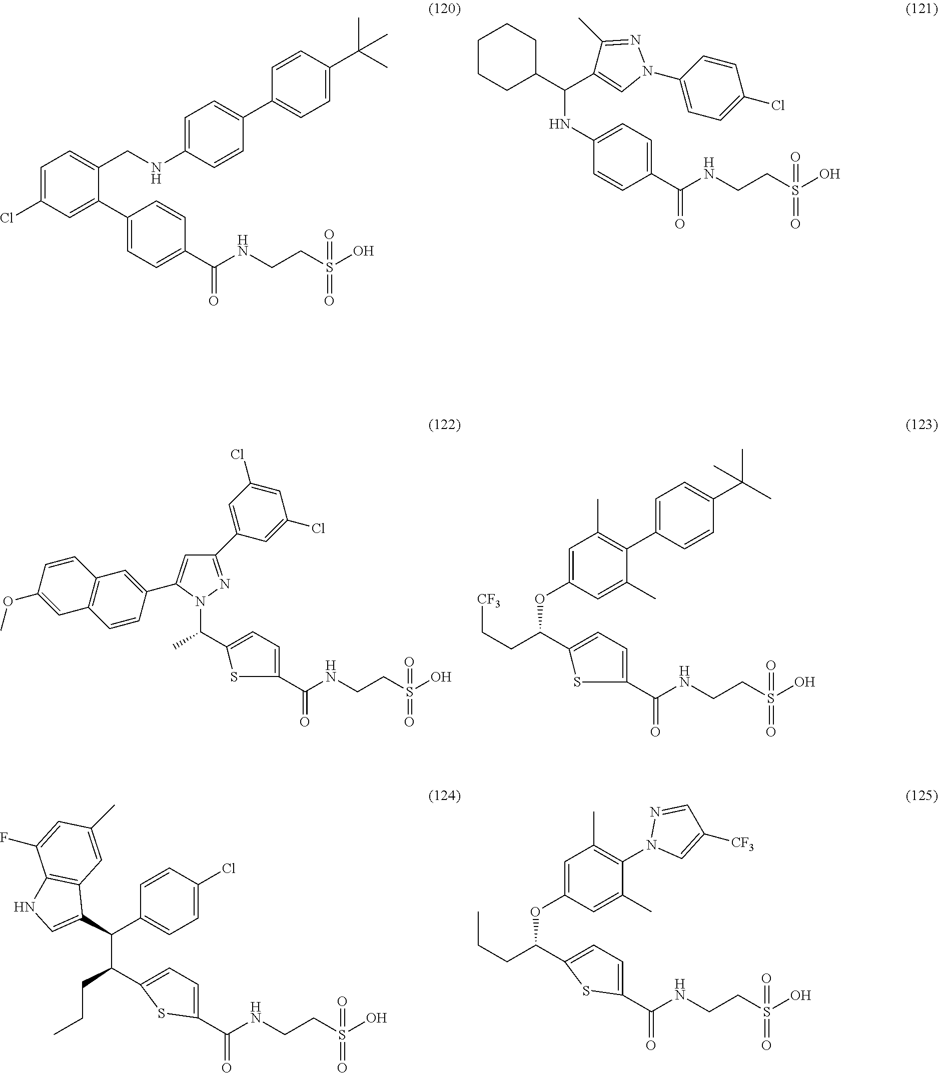 [SMILES: CC(C)(C)c1ccc(-c2ccc(NCc3ccc(Cl)cc3-c3ccc(C(=O)NCCS(=O)(=O)O)cc3)cc2)cc1 ; Cc1nn(-c2ccc(Cl)cc2)cc1C(Nc1ccc(C(=O)NCCS(=O)(=O)O)cc1)C1CCCCC1 ; COc1ccc2cc(-c3cc(-c4cc(Cl)cc(Cl)c4)nn3[C@@H](C)c3ccc(C(=O)NCCS(=O)(=O)O)s3)ccc2c1 ; Cc1cc(O[C@@H](CCC(F)(F)F)c2ccc(C(=O)NCCS(=O)(=O)O)s2)cc(C)c1-c1ccc(C(C)(C)C)cc1 ; CCC[C@H](c1ccc(C(=O)NCCS(=O)(=O)O)s1)[C@H](c1ccc(Cl)cc1)c1c[nH]c2c(F)cc(C)cc12 ; CCC[C@H](Oc1cc(C)c(-n2cc(C(F)(F)F)cn2)c(C)c1)c1ccc(C(=O)NCCS(=O)(=O)O)s1]
or a pharmaceutically acceptable salt, solvate, or prodrug thereof.

6. A pharmaceutical composition comprising a compound according to claim 1, and one or more pharmaceutically acceptable excipients or carriers.

7. The pharmaceutical composition of claim 6, further comprising a second therapeutic agent.

8. The pharmaceutical composition of claim 7, wherein the second therapeutic agent is an antidiabetic agent.

9. The pharmaceutical composition of claim 7, wherein the second therapeutic agent is at least one selected from the group consisting of: insulin sensitizers, biguanides, metformin, PPAR agonists, triglitazone, pioglitazone, rosiglitazone, insulin and insulin mimetics, somatostatin, I-glucosidase inhibitors, voglibose, miglitol, acarbose, dipeptidyl peptidase-4 inhibitors, SGLT-2 inhibitors, liver X receptor modulators, insulin secretagogues, acetohexamide, carbutamide, chlorpropamide, glibornuride, gliclazide, glimerpiride, glipizide, gliquidine, glisoxepid, glyburide, glyhexamide, glypinamide, phenbutamide, sulfonylureas, tolazamide, tolbutamide, tolcyclamide, nateglinide, repaglinide, other glucagon receptor antagonists, GLP-1, GLP-1 mimetics, exenatide, liraglutide, DPPIV inhibitors, GLP-1 receptor agonists, GIP, GIP mimetics, GIP receptor agonists, PACAP, PACAP mimetics, PACAP receptor 3 agonists, cholesterol lowering agents, HMG-CoA reductase inhibitors, statins, lovastatin, simvastatin, pravastatin, fluvastatin, atorvastatin, itavastatin, rivastatin, NK-104, itavastatin, nisvastatin, nisbastatin, ZD-4522 rosuvastatin, atavastatin, visastatin, a cholesterol absorption inhibitor ezetimibe, sequestrants, nicotinyl alcohol, nicotinic acid and salts thereof, PPAR α agonists, PPAR α/γ dual agonists, inhibitors of cholesterol absorption, acyl CoA:cholesterol acyltransferase inhibitors, anti-oxidants, PPAR δ agonists, antiobesity compounds, ileal bile acid transporter inhibitors, anti-inflammatory agents, and protein tyrosine phosphatase-1B (PTP-1B) inhibitors.

10. A method of treating, preventing, or ameliorating one or more symptoms of a condition, disorder, or disease responsive to the modulation of a glucagon receptor, comprising administering a compound of claim 1 to the subject.

11. A method of treating, preventing, or ameliorating one or more symptoms of a condition, disorder, or disease responsive to the modulation of a glucagon receptor, comprising administering any of the pharmaceutical compositions of claim 6 to the subject.

12. A method of treating, preventing, or ameliorating one or more symptoms of a condition, disorder, or disease responsive to a decrease in the hepatic glucose production or in the blood glucose level of a subject, comprising administering a compound of claim 1 to the subject.

13. A method of treating, preventing, or ameliorating one or more symptoms of a condition, disorder, or disease responsive to a decrease in the hepatic glucose production or in the blood glucose level of a subject, comprising administering any of the pharmaceutical compositions of claim 6 to the subject.

14. A method of treating, preventing, or ameliorating one or more symptoms of at least one condition, disorder or disease in a subject selected from the group consisting of type 1 diabetes, type 2 diabetes, gestational diabetes, ketoacidosis, nonketotic hyperosmolar coma, nonketotic hyperglycemia, impaired glucose tolerance (IGT), insulin resistance syndromes, syndrome X, low HDL levels, high LDL levels, hyperglycemia, hyperinsulinemia, hyperlipidemia, hypertriglyceridemia, hyperlipoproteinemia, hypercholesterolemia, dyslipidemia, arteriosclerosis, atherosclerosis, glucagonomas, acute pancreatitis, cardiovascular diseases, hypertension, cardiac hypertrophy, gastrointestinal disorders, obesity, vascular restenosis, pancreatitis, neurodegenerative disease, retinopathy, nephropathy, neuropathy, accelerated gluconeogenesis, excessive or greater than normal levels of hepatic glucose output, and lipid disorders, comprising administering a compound according to claim 1 to the subject.

15. A method of treating, preventing, or ameliorating one or more symptoms of at least one condition, disorder or disease in a subject selected from the group consisting of type 1 diabetes, type 2 diabetes, gestational diabetes, ketoacidosis, nonketotic hyperosmolar coma, nonketotic hyperglycemia, impaired glucose tolerance (IGT), insulin resistance syndromes, syndrome X, low HDL levels, high LDL levels, hyperglycemia, hyperinsulinemia, hyperlipidemia, hypertriglyceridemia, hyperlipoproteinemia, hypercholesterolemia, dyslipidemia, arteriosclerosis, atherosclerosis, glucagonomas, acute pancreatitis, cardiovascular diseases, hypertension, cardiac hypertrophy, gastrointestinal disorders, obesity, vascular restenosis, pancreatitis, neurodegenerative disease, retinopathy, nephropathy, neuropathy, accelerated gluconeogenesis, excessive or greater than normal levels of hepatic glucose output, and lipid disorders, comprising administering a compound according to claim 6 to the subject.

16. The method of claim 10, wherein the disease is diabetes.

17. The method of claim 10, wherein the disease is type II diabetes.

18. The method of claim 10, wherein the subject is a mammal.

19. The method of claim 10, wherein the subject is human.

20. A compound of Formula I, or a pharmaceutically acceptable salt, solvate, or prodrug thereof, for use in treating, preventing, or ameliorating one or more symptoms of a condition, disorder, or disease responsive to a decrease in the hepatic glucose production or in the blood glucose level of a subject, 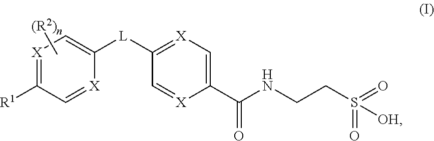
wherein:
R¹ is selected from the group consisting of hydrogen, halogen, an optionally substituted C₁₋₆-alkyl, an optionally substituted C₁₋₆-heteroalkyl, an optionally substituted C₂₋₆-alkenyl, an optionally substituted C₁₋₆-heteroalkenyl, an optionally substituted aryl, and an optionally substituted heteroaryl;
R² is selected from the group consisting of hydrogen, halogen, and an optionally substituted C₁₋₆-alkyl;
R³ is selected from the group consisting of hydrogen, an optionally substituted C₁₋₆-alkyl, and an optionally substituted C₁₋₆-heteroalkyl;
X is independently CH or N;
L is selected from a group consisting of —OCHR³—, —SCHR³—, —NR³CHR³—, —CHR¹CHR³—, —CHR³O, —CHR³S, —CHR³NR³—, arylCHR³—, -arylO—, -heteroarylCHR³—, -heteroarylCHR³NH—, -heteroarylCHR³O—, -heteroarylO—, —CHR³aryl-, —CHR³heteroaryl-, —OCH₂CHR³—, —NHCHR³aryl-, —OCHR³aryl-, and —NHCHR³heteroaryl-, where the aryl and the heteroaryl are independently optionally substituted;
n is 1, 2, or 3.

21. The compound for use of claim 20, wherein:
R² is H or Cl or CH₃;
R³ is selected from a group of hydrogen, CH₃, CH₂CH₃, CH₂CH₂CH₃, and CH₂CH₂CF₃; X is CH;
L is —OCHR³—, —SCHR³—, —NR³CHR³—, —CHR¹CHR³—, —CHR³O—, —CHR³S—, —CHR³NR³—, -heteroarylCHR³—, -heteroarylO—, —OCH₂CHR³—, —NHCHR³aryl-, and —OCHR³aryl-, where the aryl and the heteroaryl are independently optionally substituted.

22. The compound for use of claim 20, wherein L is —OCHR3—, —CHR1CHR3, and -heteroarylCHR3—, where the heteroaryl is substituted with an aryl optionally substituted with F, Cl, CH3, OCH3, and CF₃.

23. A compound of Formula II, or a pharmaceutically acceptable salt, solvate, or prodrug thereof, for use in treating, preventing, or ameliorating one or more symptoms of a condition, disorder, or disease responsive to a decrease in the hepatic glucose production or in the blood glucose level of a subject, 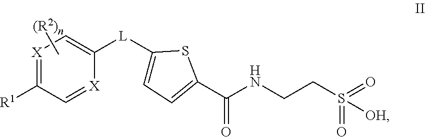
wherein:
R¹ is selected from the group consisting of hydrogen, halogen, an optionally substituted C₁₋₆-alkyl, an optionally substituted C₁₋₆-heteroalkyl, an optionally substituted C₂₋₆-alkenyl, an optionally substituted C₁₋₆-heteroalkenyl, an optionally substituted aryl, and an optionally substituted heteroaryl;
R² is selected from the group consisting of hydrogen, halogen, and an optionally substituted C₁₋₆-alkyl;
R³ is selected from the group consisting of hydrogen, an optionally substituted C₁₋₆-alkyl, and an optionally substituted C₁₋₆-heteroalkyl;
X is independently CH or N;
L is selected from a group consisting of —OCHR³—, —SCHR³—, —NR³CHR³—, —CHR¹CHR³—, —CHR³O—, —CHR³S—, —CHR³NR³—, -arylCHR³—, -arylO—, -heteroarylCHR³—, -heteroarylCHR³NH—, -heteroarylCHR³O—, -heteroarylO—, —CHR³aryl-, —CHR³heteroaryl-, —OCH₂CHR³—, —NHCHR³aryl-, —OCHR³aryl-, and —NHCHR³heteroaryl-, where the aryl and the heteroaryl are independently optionally substituted;
n is 1, 2, or 3.

24. The compound for use of claim 23, wherein:
R² is H or Cl or CH₃;
R³ is selected from a group of hydrogen, CH₃, CH₂CH₃, CH₂CH₂CH₃, and CH₂CH₂CF₃; X is CH;
L is —OCHR³—, —SCHR³—, —NR³CHR³—, —CHR¹CHR³—, —CHR³O—, —CHR³S—, —CHR³NR³—, -heteroarylCHR³—, -heteroarylO—, —OCH₂CHR³—, —NHCHR³aryl-, and —OCHR³aryl-, where the aryl and the heteroaryl are independently optionally substituted.

25. The compound for use of claim 23, wherein L is —OCHR3—, —CHR1CHR3—, and -heteroarylCHR3—, where the heteroaryl is substituted with an aryl optionally substituted with F, Cl, CH₃, OCH3, and CF₃.

26. A compound selected from Formula (101) to (125), or a pharmaceutically acceptable salt, solvate, or prodrug thereof, for use in treating, preventing, or ameliorating one or more symptoms of a condition, disorder, or disease responsive to a decrease in the hepatic glucose production or in the blood glucose level of a subject:

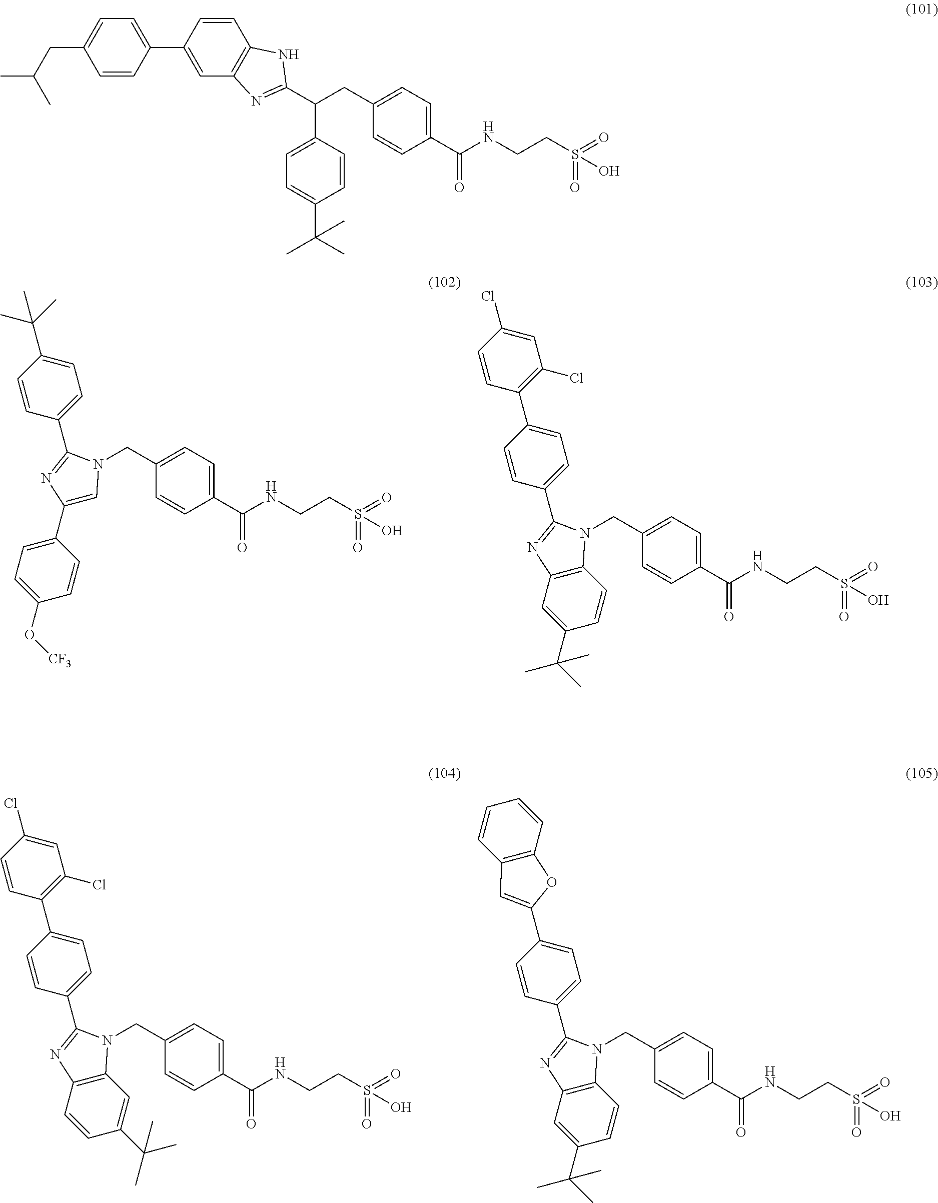 [SMILES: CC(C)Cc1ccc(-c2ccc3[nH]c(C(Cc4ccc(C(=O)NCCS(=O)(=O)O)cc4)c4ccc(C(C)(C)C)cc4)nc3c2)cc1 ; CC(C)(C)c1ccc(-c2nc(-c3ccc(OC(F)(F)F)cc3)cn2Cc2ccc(C(=O)NCCS(=O)(=O)O)cc2)cc1 ; CC(C)(C)c1ccc2c(c1)nc(-c1ccc(-c3ccc(Cl)cc3Cl)cc1)n2Cc1ccc(C(=O)NCCS(=O)(=O)O)cc1 ; CC(C)(C)c1ccc2nc(-c3ccc(-c4ccc(Cl)cc4Cl)cc3)n(Cc3ccc(C(=O)NCCS(=O)(=O)O)cc3)c2c1 ; CC(C)(C)c1ccc2c(c1)nc(-c1ccc(-c3cc4ccccc4o3)cc1)n2Cc1ccc(C(=O)NCCS(=O)(=O)O)cc1]

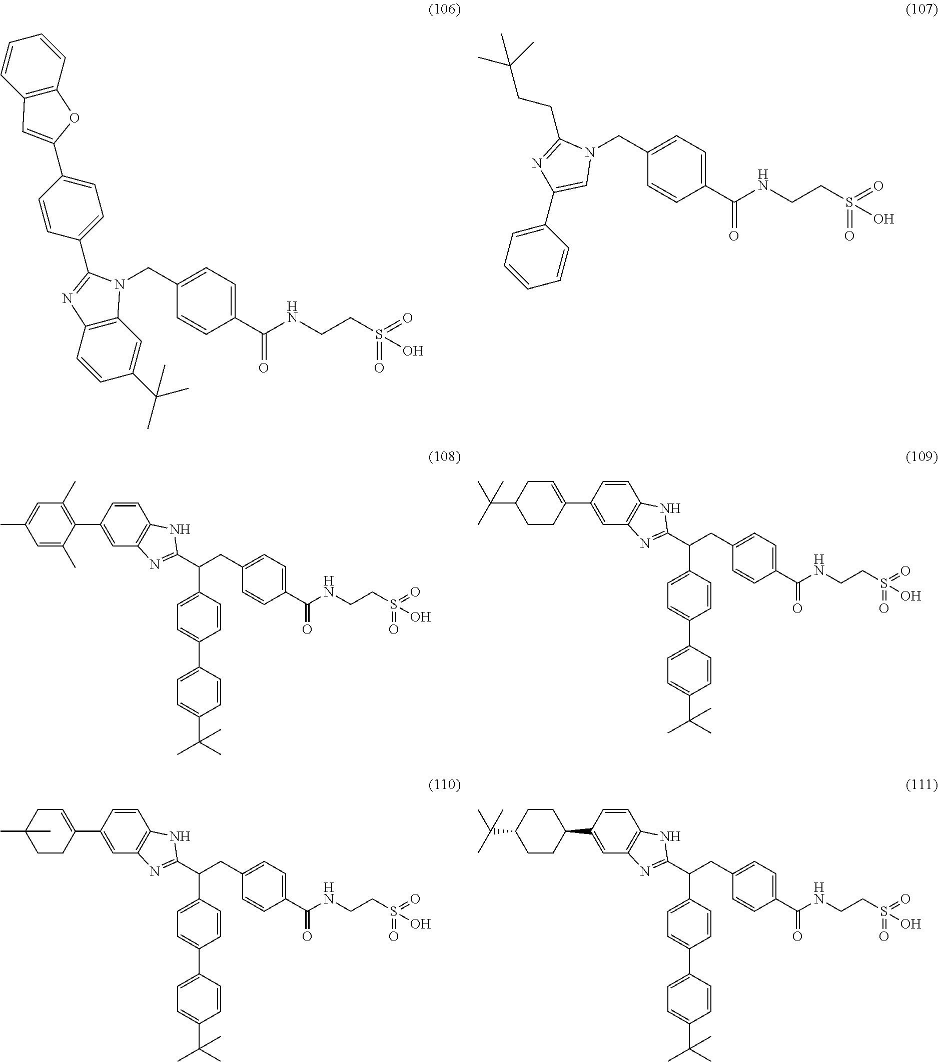

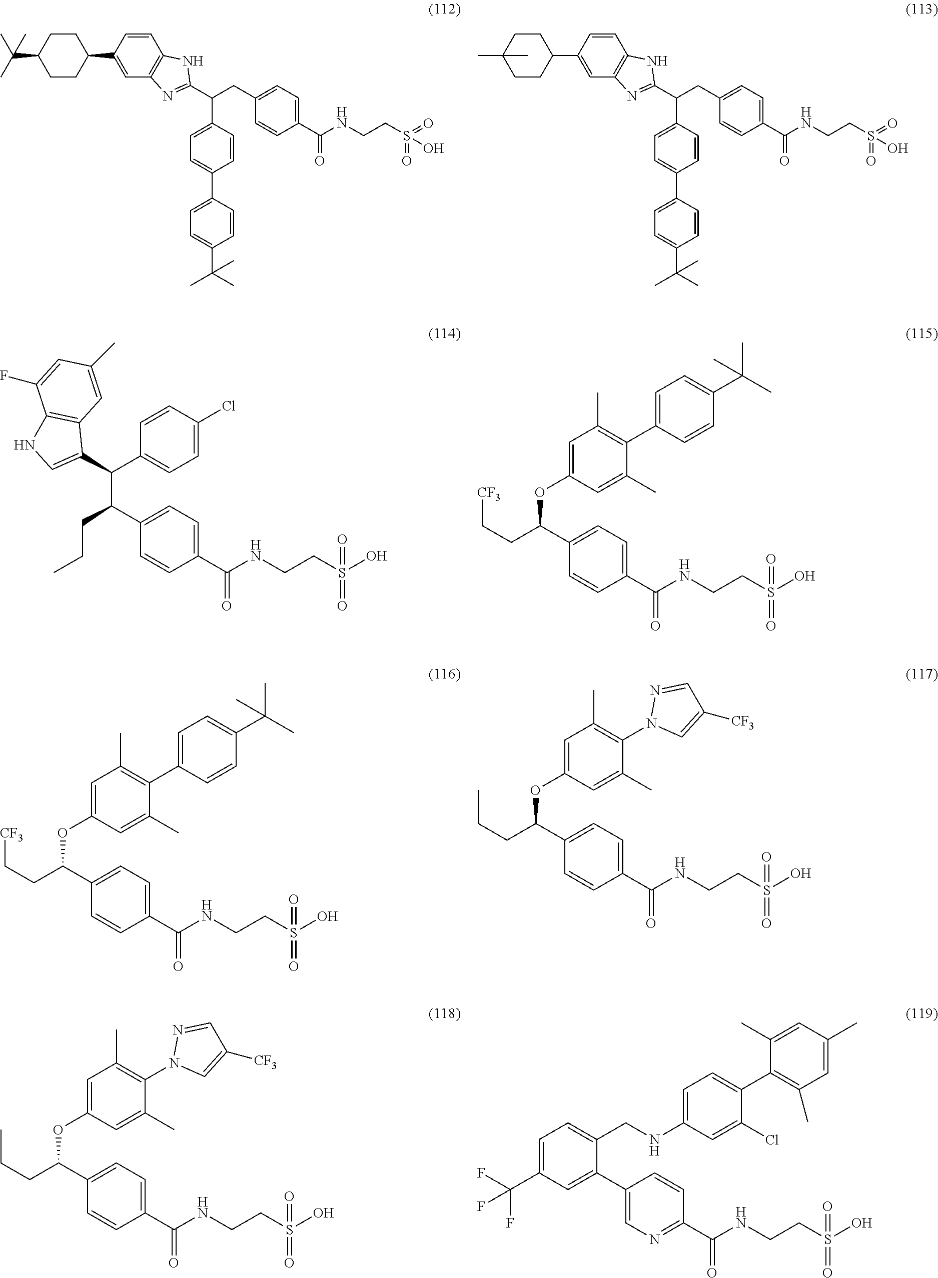

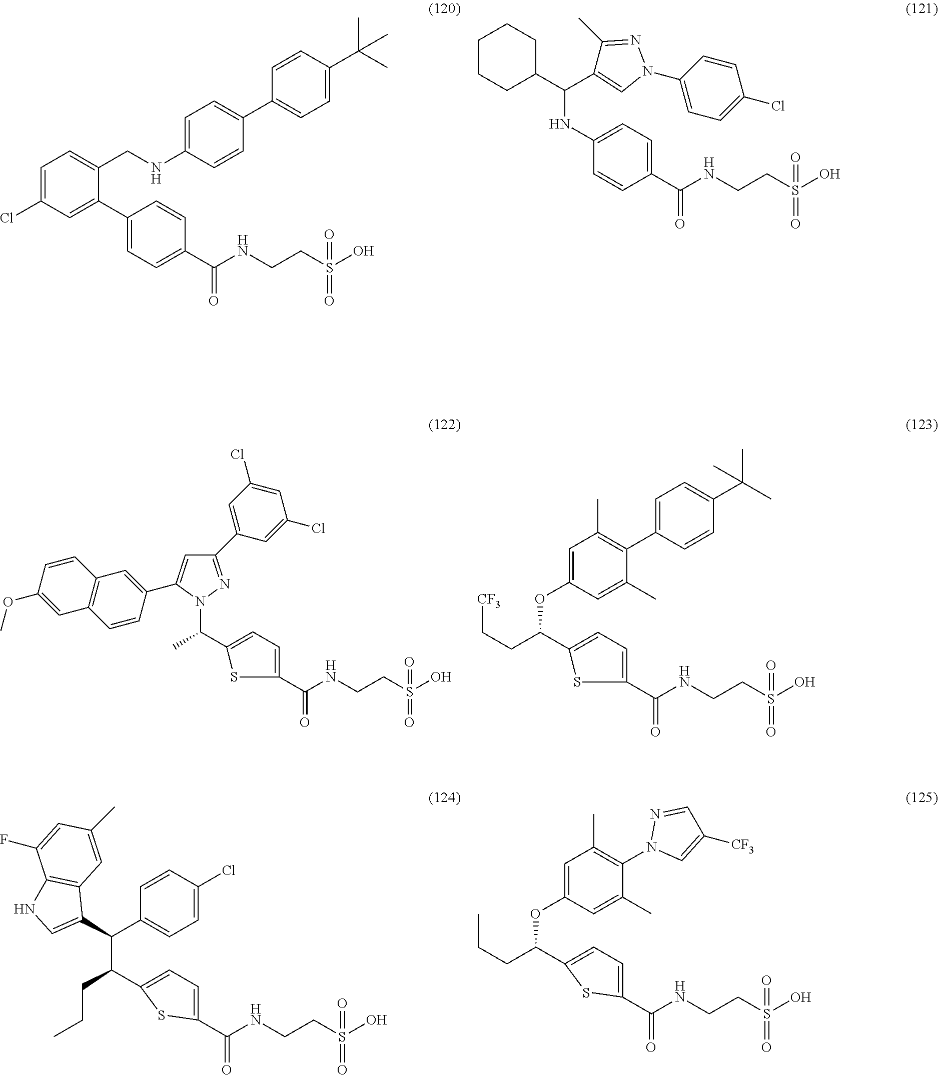 [SMILES: CC(C)(C)c1ccc(-c2ccc(NCc3ccc(Cl)cc3-c3ccc(C(=O)NCCS(=O)(=O)O)cc3)cc2)cc1 ; Cc1nn(-c2ccc(Cl)cc2)cc1C(Nc1ccc(C(=O)NCCS(=O)(=O)O)cc1)C1CCCCC1 ; COc1ccc2cc(-c3cc(-c4cc(Cl)cc(Cl)c4)nn3[C@@H](C)c3ccc(C(=O)NCCS(=O)(=O)O)s3)ccc2c1 ; Cc1cc(O[C@@H](CCC(F)(F)F)c2ccc(C(=O)NCCS(=O)(=O)O)s2)cc(C)c1-c1ccc(C(C)(C)C)cc1 ; CCC[C@H](c1ccc(C(=O)NCCS(=O)(=O)O)s1)[C@H](c1ccc(Cl)cc1)c1c[nH]c2c(F)cc(C)cc12 ; CCC[C@H](Oc1cc(C)c(-n2cc(C(F)(F)F)cn2)c(C)c1)c1ccc(C(=O)NCCS(=O)(=O)O)s1]

27. The compound of claim 20, wherein the disease is diabetes.

28. The compound of claim 20, wherein the disease is type II diabetes.

29. The compound of claim 20, wherein the subject is a mammal.

30. The compound of claim 20, wherein the subject is human.
